(19) United States
(12) Patent Application Publication
(10) Pub. No.: US 2012/0021042 A1
(43) Pub. Date: Jan. 26, 2012

(54) Efficient Method For Loading Amphoteric Liposomes With Nucleic Acid Active Substances

(76) Inventors: Steffen Panzner, Halle (DE); Gerold Endert, Halle (DE); Una Rauchhaus, Halle (DE); Natalie Herzog, Cottbus (DE); Claudia Muller, Nerchau (DE)

(21) Appl. No.: 12/225,030

(22) PCT Filed: Mar. 16, 2007
(86) PCT No.: PCT/EP2007/002349

§ 371 Date: Sep. 12, 2008

Related U.S. Application Data
(63) Continuation of application No. 11/521,857, filed on Sep. 15, 2006, now abandoned.
Application No. PCT/EP2007/002349 is a continuation of application No. 11/581,054, filed on Oct. 13, 2006, now abandoned.
(60) Provisional application No. 60/717,199, filed on Sep. 15, 2005.
(60) Provisional application No. 60/717,291, filed on Sep. 15, 2005.

(30) Foreign Application Priority Data
Mar. 17, 2006 (DE) 10 2006 013 121.5
May 10, 2006 (EP) 06113784.0
Sep. 15, 2006 (EP) 06254821.9
Sep. 15, 2006 (EP) PCT2006 009013
Oct. 13, 2006 (EP) 06255277.3
Nov. 10, 2006 (DE) 10 2006 054 192.8

(51) Int. Cl. A61K 9/127; A61K 31/713; A61K 38/02; A61K 48/00; C12N 5/00

(52) U.S. Cl. 424/450; 514/44 R; 435/375; 514/20.9; 514/44 A; 264/4.1

(57) ABSTRACT

A method for preparing amphoteric liposomes loaded with polyanionic active agent as cargo, characterised by admixing an aqueous solution of said polyanionic active agent and an alcoholic solution of one or more amphiphiles and buffering said admixture to an acidic pH, said one or more amphiphiles being susceptible of forming amphoteric liposomes at said acidic pH, thereby to form such amphoteric liposomes in suspension encapsulating said active agent under conditions such that said liposomes form aggregates, and thereafter treating said suspension to dissociate said aggregates. Also disclosed are nucleic acid loaded amphoteric liposomes produced in accordance with the method, wherein said nucleic acids are oligonucleotides and said liposomes are multilamellar.

### FIELD OF THE INVENTION

The invention relates to a method for preparing amphoteric liposomes loaded with a polyanionic active agent, especially a nucleic acid, as cargo. The invention also provides nucleic acid-loaded amphoteric liposomes.

### BACKGROUND TO THE INVENTION

### Preparation of Liposomes

Liposomes can be prepared using a variety of methods. Such methods include the long-known lipid film procedure disclosed e.g. in U.S. Pat. No. 5,648,090 (Rahman et al.). The lipids are dissolved in an organic solvent which is subsequently removed using a rotary evaporator. The thin film being formed is added with an aqueous solution of active substance, and the lipid film is rehydrated, thereby forming liposomes which enclose part of the active substance in correspondence with their inner volume. The multilamellar particles formed in this process can be adjusted to smaller size and unilamellarity by subsequent extrusion (Olson et al., Biochim. Biophys. Acta 1979 Oct. 19, 557(1), 9-23). Particularly in the production of pharmaceutical preparations, controlling liposome size and lamellarity is of importance. During extrusion, the multilamellar liposomes are forced through membranes of well-defined pore size under high pressure, thereby obtaining the desired diameter. In a similar way as in the high-pressure homogenization described below, several cycles must be passed in order to obtain a narrow size distribution. One drawback of this method is the use of a membrane, i.e., a sensitive component subject to wear, which must be replaced at regular intervals. Membrane tearing may give rise to a loss of quality and product. Moreover, providing a lipid film for larger production batches is technically complex.

Well-known high-pressure homogenization, microfluidizer and ultrasonic procedures for the preparation of liposomes utilize shear forces and cavitation effects arising during the process in order to control the size and lamellarity of the liposomes. However, these methods result in a very high local rise of pressure and temperature, so that damage might be done to sensitive lipids, e.g. unsaturated fatty acid residues or sensitive active substances such as nucleic acids. As a result, product properties such as stability and effectiveness are adversely affected.

Other methods that have been described (Papahadjopulos, U.S. Pat. No. 4,235,871, 1980; Martin et al., U.S. Pat. No. 4,752,425, 1988) achieve inclusion of active substances by injecting a water-immiscible organic solution of lipids into an aqueous solution of the material to be entrapped. When evaporating the organic solvent of the lipid in accordance with the supply thereof, spontaneous formation of liposomes takes place. This process can be continued up to high lipid concentrations. However, water-immiscible organic solvents are employed which, due to their toxicity, must be removed completely at the end of the process in complex and cost-intensive procedures.

Preparation of Liposomes Via Ethanol Injection

Liposomes can also be prepared by injecting an ethanolic lipid phase into an aqueous phase, as described for the first time by Batzri and Korn in Biophys. Biochem. Acta 298, 1015-1019 (1973). The lipid phase is introduced into a stirred aqueous phase via a fine needle, and liposomes are formed spontaneously by dilution. This method is very simple, allowing production of liposomes on an industrial scale. U.S. Pat. No. 6,843,942 and US 2004/0032037 A1 describe a device for continuous or batch production of liposomes. An aqueous solution, optionally including active substance, and an alcoholic lipid-containing solution are supplied into the system in two separate lines. The organic phase line is perpendicular to the line with the polar aqueous phase. Through an opening at the juncture of the two lines, the solutions are mixed by means of a spray valve, thereby avoiding turbulences and shear forces. Liposomes with a narrow size distribution are formed, which can be influenced by two process parameters: on the one hand, the amount of lipid and/or the amount of solvent mixed into the polar phase can be varied. On the other hand, the size distribution of the liposomes can be influenced by varying the pressure during the process. In the examples described therein the device is explained in more detail with reference to the encapsulation of recombinant human superoxide dismutase in liposomes of a defined composition. Inclusion of the protein is effected exclusively by means of the so-called “passive” process, in which the active substance dissolved in the polar phase does not interact with the lipid membranes.

U.S. Pat. No. 6,287,591 describes a method for the inclusion of charged active substances in pH-sensitive PEG liposomes. Lipids with a protonatable amino group are used as pH-sensitive components, which lipids are positively charged at acid pH values around 4 and have nearly neutral charge at a physiological pH value around 7.5. The liposomes are produced at acid pH values (e.g. 3.8), using an ethanol injection procedure. At such pH values, the negatively charged oligonucleotide active substances interact with the pH-sensitive lipids present in cationic form. By using an additional lipid component having hydrophilic polymers (e.g. PEG lipids), aggregation of the lipid particles being formed is prevented. An intermediate is formed, which is composed of active substance-containing liposomes and free active substance binding to the surface of the liposomes via ionic interactions. To obtain defined size distributions, the liposomes can be subjected to further treatment using methods well-known to those skilled in the art, such as extrusion or ultrasonic treatment. In a final step, the oligonucleotides adhering to the outside of the membrane are detached by a pH change. However, the method described requires the presence of PEG lipids in the liposomal membrane to avoid aggregation of the lipid particles during the process of formation. However, the use of PEG lipids in pharmaceutical preparations may trigger immune reactions. Ultimately, this results in a shorter circulation half-life when repeatedly administering the PEGylated liposomes.

Amphoteric Liposomes

Amphoteric liposomes represent a recently described new class of liposomes having an anionic or neutral charge at pH 7.5 and a cationic charge at pH 4. The amphoteric liposomes and the lipids suitable in the production thereof have been described in detail in WO 02/066490 A2, WO 02/066489 A2, WO 02/066012 A2, WO 03/070735 A2 and WO 03/070220 (all to Panzner et al.). Amphoteric liposomes have an excellent in vivo biodistribution and are very well tolerated by animals.

Moreover, WO 02/066012 A2 describes a method allowing effective inclusion of nucleic acid active substances in amphoteric liposomes. This method utilizes the attractive interactions of the anionically charged nucleic acid active substances with the liposomal membrane having cationic charge at acid pH values.

### OBJECTS OF THE INVENTION

An object of the present invention is to provide a method for the production of nucleic acid-loaded amphoteric liposomes for therapeutic use, which method is suitable for the production of such nucleic acid-containing amphoteric liposomes on an industrial scale and avoids the draw-backs associated with prior art methods.

Another object of the present invention is to provide process parameters allowing to control the size of the amphoteric liposomes and the active substance/lipid ratio in the final product.

A particular object of the present invention is to provide method for loading amphoteric liposomes with nucleic acids with a high loading efficiency.

Yet another object of the invention is to provide nucleic acid-loaded amphoteric liposomes.

Further advantages and objects of the present invention will be apparent from the following description of the invention.

### SUMMARY OF THE INVENTION

In accordance with one aspect of the present invention there is provided method for preparing amphoteric liposomes loaded with a polyanionic active agent as cargo, characterised by admixing an aqueous solution of said polyanionic active agent and an alcoholic solution of one or more amphiphiles and buffering said admixture to an acidic pH, said one or more amphiphiles being susceptible of forming amphoteric liposomes at said acidic pH, thereby to form such amphoteric liposomes in suspension encapsulating said active agent under conditions such that said liposomes form aggregates, and thereafter treating said suspension to dissociate said aggregates.

In some embodiments, said polyanionic active agent comprises a nucleic acid. Said nucleic acid may comprise an oligonucleotide. Alternatively, in some embodiments, said nucleic acid may comprise a plasmid, except a 7000 bp plasmid encoding luciferase.

Thus, in another aspect of the present invention, there is provided a nucleic acid loaded amphoteric liposome produced in accordance with the present invention, wherein said nucleic acid comprises an oligonucleotide and said liposome is multilamellar.

It has been found that amphoteric liposomes can be loaded effectively with nucleic acids by injecting an alcoholic lipid solution into a nucleic acid-containing polar aqueous phase at acid pH value. Said pH value may be at least one unit lower than the isoelectric point of said one or more of amphiphiles.

Surprisingly, it was found that the particle aggregates forming during this process undergo redissolution upon changing the pH value or changing the ionic strength to obtain liposomes of a well-defined size. Thus, in some embodiments, said treatment to dissociate said aggregates may comprise elevating the pH of the suspension to pH 7 or more; preferably physiological pH (about pH 7.4).

In another embodiment of the invention, a two-step process is used wherein operation initially proceeds with a higher amount of solvent. This is followed by a dilution step. For example, in some embodiments, said aqueous and alcoholic solutions may be admixed such that the resultant alcohol content is greater than about 20-25% vol. Thereafter additional aqueous media may be used to said suspension such that the alcohol content thereof is less than about 20-25% vol. Preferably; said suspension is diluted with additional aqueous media to a final concentration of alcohol of less than about 15% vol., preferably 10% vol. or less.

In the dilution step, the acid pH value can be retained at first, and the pH value can be changed in an additional step. Alternatively, the pH value can be changed in the dilution step. Furthermore, it was found that precise adjustment of both the liposome size and the drug/lipid ratio is possible via process parameters such as lipid concentration, temperature, ionic strength, alcohol (e.g., ethanol) content, mixing velocity, intial molar ratio of cationic charges (N) of the lipids to anionic charges (P) of the nucleic acid or other polyanionic active agent (N/P ratio) and by means of additives such as sucrose. Surprisingly, the way of conducting the process under conditions allowing interaction between the polyanionic active agent, such as the nucleic acids, and lipid membrane results in substantially different particle sizes compared to those in the absence of such interaction. Furthermore, is was surprising to find that the process control illustrated above avoids formation of irreversible aggregates even in the absence of PEG lipids.

In one embodiment of the invention the liposomes produced are further processed in an extrusion step. This step can be effected at acid pH value, i.e., immediately before changing the pH value to >7 or after changing the pH value to >7. Surprisingly, it was found that the extrusion step does not result in substantial release of the entrapped nucleic acid active substance, irrespective of the pH value at which the extrusion is carried out. The advantage of this embodiment is that even more narrow size distributions can be obtained. More specifically, narrowing the size distribution facilitates sterile filtration of the liposomes.

Furthermore, it was surprising to find that the liposomes produced using the method according to the invention can be frozen in a suitable medium or freeze-dried for improved storage, without releasing significant amounts of entrapped active substance. It was even more surprising to find that narrowing the size distribution of the liposomes can be obtained merely by freezing and thawing. Consequently, an optionally performed sterile filtration of the liposomes can be facilitated in this way as well.

### DETAILED DESCRIPTION OF THE INVENTION

As set forth above, U.S. Pat. No. 6,843,942 and US 2004/0032037 A1 describe a device for the continuous or batch production of active substance-containing liposomes, by means of which liposomes can be produced on an industrial scale. The specification describes “passive” inclusion of recombinant superoxide dismutase in liposomes. “Passive” inclusion means that no interactions take place between the lipids and the active substance to be entrapped, and that the inclusion efficiency is determined by the overall inner volume of the liposomes having formed.

WO 02/066012 A2 describes the production of DNA-loaded amphoteric liposomes by means of an extrusion process, utilizing the attractive interactions of the anionically charged nucleic acid active substance with the liposomal membrane having a cationic charge at acid pH value.

Now, when producing nucleic acid-loaded amphoteric liposomes under binding conditions as described in WO 02/066012 A2, using ethanol injection, it is surprising to find that the size of the resulting active substance-containing amphoteric liposomes is markedly different from the size of amphoteric liposomes produced under non-binding conditions (see “passive” inclusion) and is shifted towards larger diameters.

Furthermore, it was found that varying the process parameters gives rise to surprising effects described in more detail below, which can be utilized to control the size of nucleic acid-loaded amphoteric liposomes and the active substance/lipid ratio in the final product.

Thus, in contrast to “passive” inclusion of active substances in liposomes, it was surprising to find that the inclusion efficiency in the method according to the invention is increased at lower lipid concentrations in the process.

Moreover, and quite surprisingly, a temperature dependence of the inclusion efficiency was found in the method according to the invention. Thus, higher inclusion efficiency is achieved at elevated temperatures.

Furthermore, increasing the ionic strength and osmolarity in the aqueous solution of active substance by means of sodium chloride or other ions or salts respectively affords surprising results in the method according to the invention. For example, the inclusion efficiency decreases up to a specific NaCl concentration, while at the same time there is an increase in size of the liposomes being formed. Initially, the size of the liposomes and the inclusion efficiency remain unchanged when further increasing the NaCl concentration. Ultimately, the interaction between the amphoteric liposomes and the nucleic acids is completely suppressed at very high salt concentrations, so that inclusion proceeds via the “passive” process. This is reflected in the smaller size of the amphoteric liposomes being formed. To increase the ionic strength and osmolarity of the aqueous solution of active substance additional buffer substances and/or other ionic substances may be added.

In contrast, a different effect appears when adding a sugar (e.g. sucrose) or alternative non-ionic ingredients instead of salt to the aqueous solution of active substance in order to increase the osmolarity. Surprisingly, the size of the loaded amphoteric liposomes produced by the method according to the invention remained nearly constant, while the inclusion efficiency increased.

Another parameter that may be varied within the method of the present invention is the intial molar ratio of cationic charges (N) of the lipids to anionic charges (P) of the nucleic acid (or other polyanionic active agent) (N/P ratio). The higher the initial N/P ratio the lower the size of the resulting drug loaded amphoteric liposomes, whereas the encapsulation efficiency remains unaffected.

Furthermore the mixing velocity of the alcoholic lipid solution and the aqueous polyanionic active agent solution may be also a parameter that can be varied within the method of the present invention. For example, if nucleic acid loaded amphoteric liposomes are prepared by using an apparatus illustrated in FIG. 1 it was found that the size of the liposomes is decreased at higher volume flows.

The method according to the invention, which allows to produce polyanionic active agent-loaded amphoteric liposomes, will be described in more detail below.

Amphoteric Liposomes

In another aspect of the present invention there are provided nucleic acid loaded amphoteric liposomes prepared by the method according of the present invention.

Amphoteric liposomes can be produced from lipid mixtures comprising either an amphoteric lipid or a mixture of lipids with amphoteric properties and optionally a neutral lipid.

“Amphoteric” means that the liposomes comprise both anionic and cationic functional groups, with

(i) at least one of the charged groups having a pK between 4 and 7.4,

(ii) the cationic charge prevailing at pH 4, and

(iii) the anionic charge prevailing at pH 7.4,

the liposomes having an isoelectric point between 4 and 7.4. Amphoteric character is different from zwitterionic character, as zwitterions do not have a pK in the range mentioned above. Consequently, zwitterions are essentially uncharged over a wide range of pH values. For example, phosphatidylcholine and phosphatidylethanolamine are neutral lipids with zwitterionic character.

In some embodiments of the present invention, said amphoteric liposomes may be formed from a lipid phase comprising one or more amphoteric lipids.

Suitable amphoteric lipids are disclosed in WO 02/066489 and WO 03/070735. Preferably, said amphoteric lipid is selected from the group consisting of HistChol, HistDG, isoHistSuccDG, Acylcarnosin, HCChol, Hist-PS and EDTA-Chol.

In yet another embodiment the lipid phase may comprise a plurality of charged amphiphiles which in combination with one another have amphoteric character.

In one aspect of this embodiment said one or more charged amphiphiles comprise a pH sensitive anionic lipid and a pH sensitive cationic lipid as disclosed in WO 02/066012. Cationic pH-sensitive lipids have been described in WO 02/066489 and WO 03/070220. Moreover, Budker et al., Nat. Biotechnol. 14(6), 760-4, 1996, have described further cationic pH-sensitive lipids. Herein, such a combination of a chargeable cation and chargeable anion is referred to as an “amphoteric II” lipid pair. Suitably, said chargeable cations have pK values of between about 4 and about 8, preferably of between about 5.5 and about 7.5. Suitably, said chargeable anions have pK values of between about 3.5 and about 7, preferably of between about 4 and about 6.5. Examples include, but are not limited to MoChol/CHEMS, DPIM/CHEMS and DPIM/DGSucc.

In a second aspect of this embodiment said one or more charged amphiphiles comprise a stable cation and a chargeable anion and is referred to as “amphoteric I” lipid pair. Examples include, without limited to DDAB/CHEMS, DOTAP/CHEMS and DOTAP/DMG-Succ.

In a third aspect of this embodiment said one or more charged amphiphiles comprise a stable anion and a chargeable cation and is referred to as “amphoteric III” lipid pair. Examples include, but not limited to MoChol/DOPG and MoChol/Choi-SO4.

It is of course possible to use amphiphiles with multiple charges such as amphipathic dicarboxylic acids, phosphatidic acid, amphipathic piperazine derivatives and the like. Such multicharged amphiphiles might fall into pH sensitive amphiphiles or stable anions or cations or might have mixed character.

Preferred cationic components include without limitation DPIM, DOIM, CHIM, DORIE, DDAB, DAC-Chol, TC-Chol, DOTMA, DOGS, (C18)₂Gly⁺-N,N-dioctadecylamidoglycine, CTAB, CPyC, DODAP, DOEPC, DMTAP, DPTAP, DOTAP, DC-Chol, MoChol and HisChol.

Particularly preferred cationic lipids comprise DMTAP, DPTAP, DOTAP, DC-Chol, MoChol, Chim and HisChol.

In addition, the amphoteric mixtures include anionic lipids either having a constitutive charge or being charged in correspondence to the pH, and these lipids are also well-known to those skilled in the art. Preferred lipids for use in this invention include but are not limited to DOGSucc, POGSucc, DMGSucc, DPGSucc, DMPS, DPPS, DOPS, POPS, DMPG, DPPG, DOPG, POPG, DMPA, DPPA, DOPA, POPA, CHEMS and Cetyl-P.

Particularly preferred anionic lipids comprise DOGSucc, DMGSucc, DMPG, DPPG, DOPG, POPG, DMPA, DPPA, DOPA, POPA, CHEMS and Cetyl-P.

In one embodiment of the invention the above-mentioned cationic lipids may comprise one or more of the following lipids: DOTAP, DC-Chol, CHIM, MoChol and HisChol. The above-mentioned anionic lipids may include one or more of the following lipids: DMGSucc, DOGSucc, DOPA, CHEMS and CetylP.

In further embodiments of the invention neutral lipids may be present in the amphoteric lipid mixtures as well. Said neutral lipids comprise but are not limited to natural or synthetic phosphatidylcholines, phosphatidylethanolamines, sphingolipids, ceramides, cerebrosides, sterolbased lipids, e.g. cholesterol, and derivatives of such lipids. Specific examples of neutral lipids include, without limited to DMPC, DPPC, DSPC, POPC, DOPC, DMPE, DPPE, DSPE, POPE, DOPE, Diphythanoyl-PE, sphingomyelein, ceramide and cholesterol. Of course, mixtures of neutral lipids in the amphoteric lipid mixtures are also within the scope of the present invention.

In a preferred embodiment the phosphatidylcholines are selected from the following group: POPC, natural or hydrogenated soy bean PC, natural or hydrogenated egg PC, DMPC, DPPC or DOPC. (See below for a list of abbreviations of the lipids being used and of the names thereof. In many cases, the abbreviations commonly used in the literature will be used).

Particularly preferred phosphatidylcholines are DMPC, POPC, DOPC, non-hydrogenated soy bean PC and non-hydrogenated egg PC.

The phosphatidylethanolamines can be selected from the group of DOPE, DMPE and DPPE.

In a preferred fashion the above-mentioned neutral lipids comprise DOPE, DMPC, POPC, DOPC, non-hydrogenated soy bean PC and non-hydrogenated egg PC.

In another embodiment the lipid mixture of said amphoteric lipids may also include the neutral lipid cholesterol and derivatives thereof.

In addition, the amphoteric mixtures according to the present invention may include known fusogenic lipids, such as for example DOPE, lysolipids or free fatty acids or mixtures of said fusogenic lipids.

The following list shows the abbreviations of the lipids being used as well as the names thereof. In many cases, the abbreviations commonly used in the literature will be used.

DMPC Dimyristoylphosphatidylcholine

DPPC Dipalmitoylphosphatidylcholine

DSPC Distearoylphosphatidylcholine

POPC Palmitoyloleoylphosphatidylcholine

DOPC Dioleoylphosphatidylcholine

DOPE Dioleoylphosphatidylethanolamine

DMPE Dimyristoylphosphatidylethanolamine

DPPE Dipalmitoylphosphatidylethanolamine

Diphytanoyl-PE Diphytanoylphosphatidylethanolamine

DOPG Dioleoylphosphatidylglycerol

POPG Palmitoyloleoylphosphatidylglycerol

DMPG Dimyristoylphosphatidylglycerol

DPPG Dipalmitoylphosphatidylglycerol

DMPS Dimyristoylphosphatidylserine

DPPS Dipalmitoylphosphatidylserine

DOPS Dioleoylphosphatidylserine

POPS Palmitoyl-oleoylphosphatidylserine

DMPA Dimyristoylphosphatidic acid

DPPA Dipalmitoylphosphatidic acid

DOPA Dioleoylphosphatidic acid

POPA Palmitoyl-oleoylphosphatidic acid

DMPI Dimyristoylphosphatidylinositol

DPPI Dipalmitoylphosphatidylinositol

DOPI Dioleoylphosphatidylinositol

POPI Palmitoyloleoylphosphatidylinositol

CHEMS Cholesterol hemisuccinate

Chol-Sulfate Cholesterolsulfate

Chol-Phosphate Cholesterolphosphate

DC-Chol 3-β-[N—(N′,N′-dimethylethane)carbamoyl]cholesterol

CetylP Cetyl phosphate

DODAP (1,2-dioleoyloxypropyl)-N,N-dimethylammonium chloride

DOEPC 1,2-dioleoyl-sn-glycero-3-ethylphosphocholine

DAC-Chol 3-β-[N—(N,N′-dimethylethane)carbamoyl]cholesterol

TC-Chol 3-β-(N—(N′,N′,N′-trimethylaminoethane)carbamoyl)cholesterol

DOTMA (1,2-dioleyloxypropyl)-N,N,N-trimethylammonium chloride (Lipofectin®)

DOGS ((C18)₂GlySper3⁺)N,N-dioctadecylamido-glycyl-spermine (Transfectam®)

CTAB Cetyltrimethylammonium bromide

CPyC Cetylpyridinium chloride

DOTAP (1,2-dioleoyloxypropyl)-N,N,N-trimethylammonium salt

DMTAP (1,2-dimyristoyloxypropyl)-N,N,N-trimethylammonium salt

DPTAP (1,2-dipalmitoyloxypropyl)-N,N,N-trimethylammonium salt

DOTMA (1,2-dioleyloxypropyl)-N,N,N-trimethylammonium chloride

DORIE (1,2-dioleyloxypropyl)-3-dimethylhydroxyethylammonium bromide

DDAB Dimethyldioctadecylammonium bromide

DPIM 4-(2,3-bis-palmitoyloxy-propyl)-1-methyl-1H-imidazole

DOIM 4-(2,3-bis-oleoyloxy-propyl)-1-methyl-1H-imidazole

CHIM Histaminylcholesterol carbamate

MoChol 4-(2-Aminoethyl)morpholinocholesterol hemisuccinate

HisChol Histaminylcholesterol hemisuccinate

HCChol Nα-Histidinylcholesterol carbamate

HistChol Nα-Histidinylcholesterol hemisuccinate

AC Acylcarnosine, stearyl- & palmitoylcarnosine

HistDG 1,2-Dipalmitoylglycerol hemisuccinate, N-Histidinyl hemisuccinate & distearoyl, dimyristoyl, dioleoyl or palmitoyl oleoyl derivatives

IsoHistSuccDG 1,2-Dipalmitoylglycerol-O-histidinyl-Nα-hemisuccinate & distearoyl, dimyristoyl, dioleoyl or palmitoyl oleoyl derivatives

DGSucc 1,2-Dipalmitoyglycerol-3-hemisuccinate & distearoyl, dimyristoyl, dioleoyl or palmitoyl oleoyl derivatives

EDTA-Chol Cholesterol ester of ethylenediaminetetraacetic acid

Hist-PS Nα-Histidinylphosphatidylserine

BGSC Bisguanidinium-spermidinecholesterol

BGTC Bisguanidinium-tren-cholesterol

DOSPER (1,3-Dioleoyloxy-2-(6-carboxyspermyl)propylamide

DOSC (1,2-Dioleovl-3-succinyl-sn-glycerylcholine ester)

DOGSDO (1,2-Dioleoyl-sn-glycero-3-succinyl-2-hydroxyethyl disulfide ornithine)

DOGSucc 1,2-Dioleoylglycerol-3-hemisucinate

POGSucc 1,2-Palmitoyl-oleoylglycerol-3-hemisuccinate

DMGSucc 1,2-Dimyristoylglycerol-3-hemisuccinate

DPGSucc 1,2-Dipalmitoylglycerol-3-hemisuccinate

The following table exemplifies non-limiting lipids suitable for the production of amphoteric liposomes of the present invention. The membrane anchors of the lipids are merely shown by way of example and are not limited to these membrane anchors.

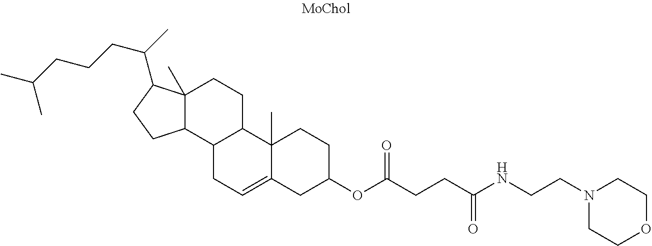

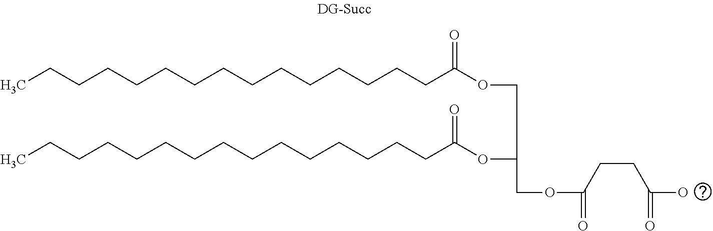

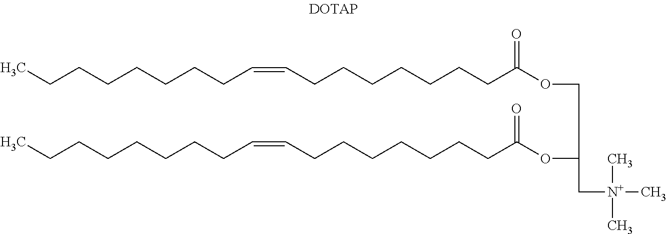

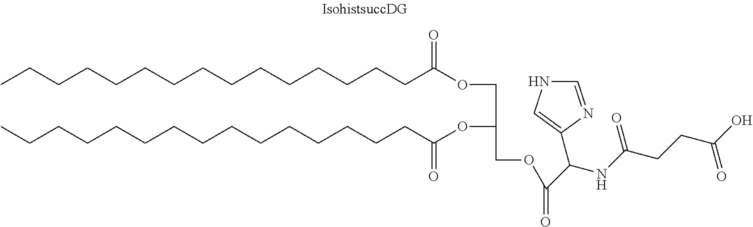

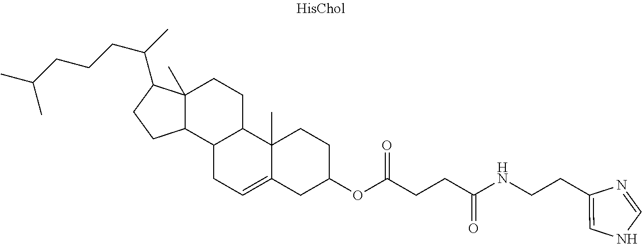

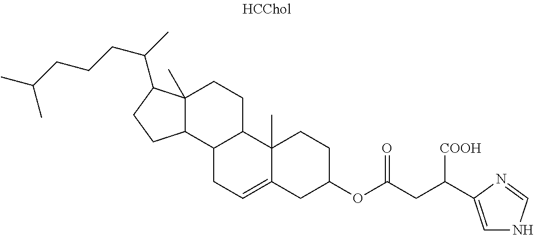

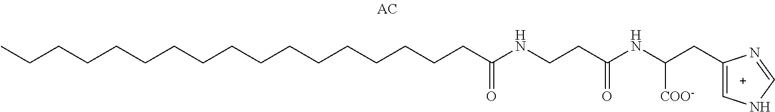

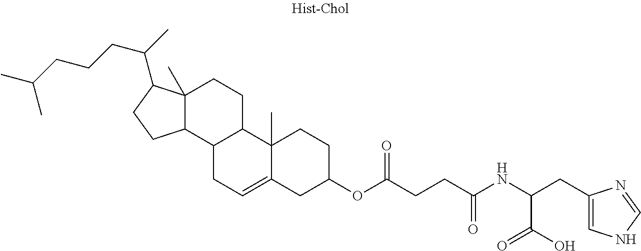

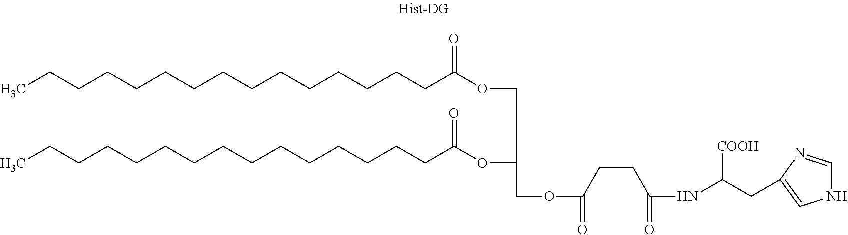

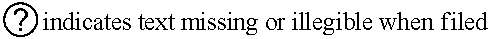

Nucleic Acids

The present invention describes a process by means of which nucleic acid-loaded amphoteric liposomes can be produced. Nucleic acids are an example of a polyanionic active agent.

Nucleic acids can be classified into nucleic acids encoding one or more specific sequences for proteins, polypeptides or RNAs, and into oligonucleotides that can specifically regulate protein expression levels or affect the protein structure through inter alia interference with splicing and artificial truncation.

In some embodiments of the present invention, therefore, the nucleic acid-based therapeutic may comprise a nucleic acid that is capable of being transcribed in a vertebrate cell into one or more RNAs, which RNAs may be mRNAs, shRNAs, miRNAs or ribozymes, wherein such mRNAs code for one or more proteins or polypeptides. Such nucleic acid therapeutics may be circular DNA plasmids, linear DNA constructs, like MIDGE vectors (Minimalistic Immunogenically Defined Gene Expression) as disclosed in WO 98/21322 or DE 19753182, or mRNAs ready for translation (e.g., EP 1392341).

In another embodiment of the invention, oligonucleotides may be used that can target existing intracellular nucleic acids or proteins. Said nucleic acids may code for a specific gene, such that said oligonucleotide is adapted to attenuate or modulate transcription, modify the processing of the transcript or otherwise interfere with the expression of the protein. The term “target nucleic acid” encompasses DNA encoding a specific gene, as well as all RNAs derived from such DNA, being pre-mRNA or mRNA. A specific hybridisation between the target nucleic acid and one or more oligonucleotides directed against such sequences may result in an inhibition or modulation of protein expression. To achieve such specific targeting, the oligonucleotide should suitably comprise a continuous stretch of nucleotides that is substantially complementary to the sequence of the target nucleic acid.

Oligonucleotides fulfilling the abovementioned criteria may be built with a number of different chemistries and topologies. Oligonucleotides may be single stranded or double stranded.

Oligonucleotides are polyanionic structures having 8-60 charges. In most cases these structures are polymers comprising nucleotides. The present invention is not limited to a particular mechanism of action of the oligonucleotides and an understanding of the mechanism is not necessary to practice the present invention.

The mechanisms of action of oligonucleotides may vary and might comprise effects on inter alia splicing, transcription, nuclear-cytoplasmic transport and translation.

In a preferred embodiment of the invention single stranded oligonucleotides may be used, including, but not limited to, DNA-based oligonucleotides, locked nucleic acids, 2′-modified oligonucleotides and others, commonly known as antisense oligonucleotides. Backbone or base or sugar modifications may include, but are not limited to, Phosphothioate DNA (PTO), 2′O-methyl RNA (2′Ome), 2′Fluoro RNA (2′F), 2′ Omethoxyethyl-RNA (2′MOE), peptide nucleic acids (PNA), N3′-P5′ phosphoamidates (NP), 2′ fluoroarabino nucleic acids (FANA), locked nucleic acids (LNA), Morpholine phosphoamidate (Morpholino), Cyclohexene nucleic acid (CeNA), tricyclo-DNA (tcDNA) and others. Moreover, mixed chemistries are known in the art, being constructed from more than a single nucleotide species as copolymers, block-copolymers or gapmers or in other arrangements. In addition to the aforementioned oligonucleotides, protein expression can also be inhibited using double stranded RNA molecules containing the complementary sequence motifs. Such RNA molecules are known as siRNA molecules, in the art (e.g., WO 99/32619 or WO 02/55693). Other siRNAs comprise single stranded siRNAs or double stranded siRNAs having one non-continuous strand. Again, various chemistries were adapted to this class of oligonucleotides. Also, DNA/RNA hybrid systems are known in the art.

In another embodiment of the present invention, decoy oligonucleotides can be used. These double stranded DNA molecules and chemical modifications thereof do not target nucleic acids but transcription factors. This means that decoy oligonucleotides bind sequence-specific DNA-binding proteins and interfere with the transcription (e.g., Cho-Chung, et al. in Curr. Opin. Mol. Ther., 1999).

In a further embodiment of the invention, oligonucleotides that may influence transcription by hybridizing under physiological conditions to the promoter region of a gene may be used. Again various chemistries may adapt to this class of oligonucleotides.

In a still further alternative of the invention, DNAzymes may be used. DNAzymes are single-stranded oligonucleotides and chemical modifications thereof with enzymatic activity. Typical DNAzymes, known as the “10-23” model, are capable of cleaving single-stranded RNA at specific sites under physiological conditions. The 10-23 model of DNAzymes has a catalytic domain of 15 highly conserved deoxyribonucleotides, flanked by 2 substrate-recognition domains complementary to a target sequence on the RNA. Cleavage of the target mRNAs may result in their destruction and the DNAzymes recycle and cleave multiple substrates.

In yet another embodiment of the invention, ribozymes can be used. Ribozymes are single-stranded oligoribonucleotides and chemical modifications thereof with enzymatic activity. They can be operationally divided into two components, a conserved stem-loop structure forming the catalytic core and flanking sequences which are reverse complementary to sequences surrounding the target site in a given RNA transcript. Flanking sequences may confer specificity and may generally constitute 14-16 nt in total, extending on both sides of the target site selected.

In a still further embodiment of the invention, aptamers may be used to target proteins. Aptamers are macromolecules composed of nucleic acids, such as RNA or DNA, and chemical modifications thereof that bind tightly to a specific molecular target and are typically 15-60 nt long. The chain of nucleotides may form intramolecular interactions that fold the molecule into a complex three-dimensional shape. The shape of the aptamer allows it to bind tightly against the surface of its target molecule including but not limited to acidic proteins, basic proteins, membrane proteins, transcription factors and enzymes. Binding of aptamer molecules may influence the function of a target molecule.

The oligonucleotides or polynucleotides specifically used are not critical to the feasibility of the method according to the invention, and also, specific modifications to the nucleobases, nucleoside sugars or the nucleic acid backbone are not required or would not impede the implementation of the method according to the invention beyond the extent of skillful optimization. The nucleic acids merely have to possess an overall anionic charge.

All of the above-mentioned oligonucleotides may vary in length between as little as 5, preferably between 8 and 50 nucleotides. The fit between the oligonucleotide and the target sequence is preferably perfect with each base of the oligonucleotide forming a base pair with its complementary base on the target nucleic acid over a continuous stretch of the abovementioned number of oligonucleotides. The pair of sequences may contain one or more mismatches within the said continuous stretch of base pairs, although this is less preferred. In general the type and chemical composition of such nucleic acids is of little impact for the performance of the invention or the use of the nucleic acid loaded amphoteric liposomes as vehicles be it in vivo or in vitro and the skilled artisan may find other types of oligonucleotides or nucleic acids suitable for the present invention.

Procedure

The method according to the invention comprises the following steps:

a) providing an optionally sterile solution of a lipid mixture in a water-miscible solvent, preferably an alcohol, which can optionally be acidified;

b) providing an optionally sterile aqueous solution of the polyanionic active agent (e.g., nucleic acid) active substance to be entrapped, which can optionally be acidified;

at least one of the two solutions in a) and b) requiring adjustment to an acid pH value by means of acids and/or buffer substances;

c) mixing defined quantities of the two solutions by injecting the alcoholic solution of lipid mixture into the aqueous solution of the polyanionic active agent or vice versa, or by combining two metered separate streams of provided lipid solution and nucleic acid solutions, optionally in one or more mixers;

d) a dilution step which is optional;

e) dissipating the interaction between the amphoteric liposomes and the polyanionic active agent by increasing the pH value to >7 or by increasing the ionic strength and subsequently increasing the pH value to >7;

f) removing non-entrapped active substance and/or concentrating the liposome suspension and/or replacing the aqueous medium and/or removing the water-miscible solvent, each of these steps being independently optional;

g) sterile filtration of the active substance-containing liposomes, which is optional;

wherein an extrusion step can be effected between the steps c) and d) and/or between d) and e) and/or between e) and f) and/or between f) and g), and/or

one or more freeze-thaw cycles can be effected between the steps e) and f) and/or between f) and g), and/or

step g) can be followed by one or more freeze-thaw cycles and/or lyophilization of the active substance-containing liposomes and

all steps of the method according to the invention may be optionally performed under aseptic conditions.

The single steps will be described in more detail below.

In a preferred embodiment of the invention the water-miscible solvent used to produce the lipid solution is an alcohol. The alcohols ethanol, isopropanol, 1,2-propanediol, npropanol, butanol, pentanol, as well as ethylene glycol, propylene glycol, methanol are preferred.

In another preferred embodiment of the invention the alcohol is ethanol, propanol or isopropanol. Of course, mixtures of the above-mentioned alcohols can also be used in order to facilitate optimum solubility of all lipid components of an amphoteric system, for example.

In another embodiment of the invention the solvent can also be diluted with water as long as the respective lipid mixture retains solubility in such mixtures.

Inter alia, the solubility of the lipids depends on the temperature. Generally speaking, the higher the temperature, the higher the solubility of the lipids. When devising the process parameters, only those concentrations and process temperatures ensuring complete dissolution of the lipid mixture can be used.

The temperature range selected according to the aspects above is between 4° C. and 100° C., preferably between 10° C. and 70° C., and more preferably between 20 and 50° C.

The alcoholic lipid solutions are used in a concentration range between 1 mM and 500 mM, preferably between 5 mM and 250 mM, and most preferably between 10 mM and 150 mM.

In one embodiment of the invention the lipid solution is acidified. To acidify the lipid solution, buffer systems well-known to those skilled in the art, e.g. acetate buffers, formiate buffers, glycine buffers, maleic acid buffers, phosphate buffers or citrate buffers, can be used. Furthermore, the pH value can be adjusted using an acid (e.g. HCl, acetic acid, formic acid, maleic acid, sulfonic acid, phosphoric acid or citric acid). Pharmaceutically highly acceptable buffers or acids such as acetic acid, citric acid, HCl, phosphoric acid or glycine are preferred.

In another embodiment, the lipids being employed are used in their undissociated form, i.e., not in the form of salts. In particular, this concerns anionic and cationic lipids used to produce the amphoteric liposomes. Thus, it is advantageous to use CHEMS in the form of the free acid rather than the sodium salt. Likewise, it is advantageous to employ MoChol in the form of the free base rather than the hydrochloride. The use of lipids free of salts facilitates unambiguous addition of specific counterions in following process steps and, as a consequence, optimum stabilization of the liposomes being obtained.

In some embodiments of the invention the addition of specific counterions to the lipid phase is advantageous for the solubilization of one or more lipids in the water miscible solvent. Said counterions may comprise but are not limited to carbonate, hydrogencarbonate, formiate, acetate, propionate, butyrate, isobutyrate, ammonium, trimethylammonium, triethylammonium, triethanolammonium, trishydroxymethylaminomethanium, BIS-TRIS, immidazolium, argininium, L-argininium, phosphate, sulphate, methanesulfonate, chloride, sodium or potassium.

In one embodiment of the invention the aqueous nucleic acid solution has a pH value of <6, preferably a pH value between 3 and 5.5, and more preferably a pH value between 3.5 and 4.5. In general, the acidic pH of the aqueous nucleic acid solution should be at least one unit lower than the isoelectric point of the amphoteric lipid mixture. To adjust the pH value, buffer systems well-known to those skilled in the art, e.g. acetate buffers, formiate buffers, glycine buffers, maleic acid buffers, phosphate buffers or citrate buffers, can be used. Furthermore, the pH value can be adjusted using an acid (e.g. HCl, acetic acid, formic acid, maleic acid, sulfonic acid, phosphoric acid or citric acid). Pharmaceutically highly acceptable buffers or acids such as acetic acid, citric acid, HCl, phosphoric acid or glycine are preferred.

The concentration of the aqueous solution of said polyanionic active agent depends on the amount of cationic lipids used in the process. In a preferred embodiment of the invention the molar ratio of cationic charges of the lipids to anionic charges of the nucleic acid or other polyanionic active agent (N/P ratio) is between 1 and 10, more preferably between 1.5 and 5, and especially preferably between 2 and 4, for single- or double-stranded oligonucleotides.

For some applications of oligonucleotide loaded amphoteric liposomes products low drug/lipid ratios are required. To this end in some embodiments of the invention higher initial N/P ratios up to 35 may be preferably.

For larger single- or double-stranded nucleic acid molecules with a chain length of 50 or more nucleobases or base pairs, preferably more than 100 nucleobases or base pairs such as plasmids, aptamers or RNA molecules, the N/P ratio is preferably between 1 and 50, more preferably between 2 and 30, and especially preferably between 3 and 20.

In a specific embodiment of the method the aqueous polyanionic active agent solution is produced just a short time prior to mixing with the lipid phase by mixing two aqueous phases. In particular, this is advantageous in those cases where the polyanionic active agent solution is present in the form of a concentrate and must be diluted suitably prior to mixing with the lipid solution. This embodiment is particularly advantageous in those cases where the dissolved active substance is sensitive to the binding conditions and cannot be stored under such conditions for a long time. For example, prolonged exposition to below pH 4 can do irreversible damage to DNA, resulting in removal of purine bases from the sugar/phosphate backbone.

Obviously, the simplest way of avoiding excessive exposition of nucleic acids in acid medium is to dissolve the active substances in a neutral aqueous buffer, the addition of acid required to adjust the process pH being accomplished via the lipid in this case, i.e., the acid is added to the ethanolic lipid solution. Of course, also the alcoholic lipid solution can be acidified just a short time prior to mixing with the aqueous polyanionic active agent solutions to avoid a long time storage of the lipids under acidic conditions.

On a laboratory scale, mixing the two solutions being provided is preferably effected by injecting the alcoholic lipid solution into a stirred aqueous polyanionic active agent solution or vice versa. To produce nucleic acid-loaded amphoteric liposomes on an industrial scale, thorough mixing of the two solutions can be effected in an apparatus. A suitable apparatus for thorough mixing of the solutions will be described in more detail below. Moreover, such mixing can be performed in the apparatus disclosed in U.S. Pat. No. 6,843,942 and US 2004/0032037.

Independent of the mixing method the process of the present invention may be performed under aseptic conditions. It is an advantage of the method according to the present invention that the starting materials are solutions that can be easily sterile filtered before entering an aseptic process. Alternative methods to sterilize solutions are well known in the art and include but are not limited to irradiation, heat sterilization, chemical sterilization and/or high pressure sterilization. In a preferred embodiment of the present invention the aseptic process is performed in an apparatus as described below in more detail or an apparatus disclosed in U.S. Pat. No. 6,843,942 and US 2004/0032037. Advantageously, the lipid solution and the nucleic acid solution may be sterile filtered just before entering the process by using the pumps of the devices to pass the solutions through one or more sterile filters that are located after the pumps.

Alternatively the process may be performed in a non-aseptic way and the final product may be sterile filtered and/or sterilized by other sterilization methods as mentioned above. In a preferred embodiment of the invention the loaded amphoteric liposomes prepared by the method of the present invention are sterile filtered through one or more filters having a pore size of less than 0.5 μm, preferably less than 0.25 μm. Most preferred are one or more sterile filters having a pore size of 0.2 μm.

Of course, a sterilization of the formed liposomes at the end of the process may also be preferably in the case of an aseptic process.

In one embodiment of the invention the amount of alcoholic lipid solution mixed into the aqueous polyanionic active agent (e.g., nucleic acid) solution is preferably between 2 and 25% and more preferably between 5 and 20% of the resulting total volume.

During and following mixing the alcoholic solution of the lipid mixture with the aqueous polyanionic active agent (nucleic acid) solution the amphoteric liposomes being or having formed are present in a state of cationic charge. For this reason, the anionically charged active agents or nucleic acids interact with the cationic lipid layer, thereby allowing formation of aggregates during the process. Surprisingly, it was found that aggregate formation during liposome production, as opposed to the description in U.S. Pat. No. 6,287,591, does not have to be prevented by means of steric hindrance (e.g. by using PEG lipids). Rather, the interactions following formation of the amphoteric liposomes can be dissipated in a further step, thereby forming polyanionic active agent (nucleic acid)-loaded amphoteric liposomes with a homogeneous size distribution.

In a specific embodiment of the invention, mixing the two solutions is followed by a dilution step. In this event, the amount of alcoholic lipid solution mixed into the aqueous polyanionic active agent solution is preferably between 20 and 50%, and more preferably between 25 and 45% of the resulting total volume.

Thus, the use of higher amounts of alcohol, such as ethanol, during the process can be advantageous. It was found that amphoteric liposomes are permeable from a critical alcohol (ethanol) concentration of about 20 or 25% to 30 or 35% on by volume. For this reason, the process of mixing the two solutions is followed by a dilution step so as to furnish stable amphoteric liposomes, the alcohol (ethanol) concentration in this case being lowered to a value of less than 25%, preferably less than 15%, e.g., about 10% vol.

In one embodiment of the invention the dilution step can be performed using an aqueous solution which corresponds in its composition to the polyanionic active agent (nucleic acid) solution but has no polyanionic active agent (nucleic acid) and approximately the pH value of the mixture. Alternatively, other solutions well-known to those skilled in the art and having the appropriate acid pH value can be used in diluting.

In another advantageous embodiment of the invention, aqueous solutions are used for diluting, which alter the pH value of the present mixture in such a way that the interactions between the amphoteric liposomes and the polyanionic active agent (nucleic acids) are dissipated. The selection of the pH value will be explained in more detail below. In general, it is well-known to those skilled in the art which pH value will result upon mixing two solutions of defined composition and in which way a solution must be modified in order to obtain the desired pH value of the mixture.

Diluting is carried out in such a way that the amount of alcohol (ethanol) in the mixture is reduced to preferably <20%, more preferably between 2 and 15%, and especially preferably between 5 and 12%.

Various methods can be used to dissipate the interaction between the amphoteric liposomes and the polyanionic active agent (nucleic acids) following formation of the liposomes.

In one embodiment of the invention the interactions are dissipated by altering the pH value. As set forth above, amphoteric liposomes have anionic or neutral charge at a pH value of 7.5. Therefore, when altering the pH value in such a way that the amphoteric liposomes become anionic or neutral, the interactions between the liposomes and the polyanionic active agent (nucleic acids) will be dissipated at the same time. Surprisingly, it was found that the change in pH value is sufficient to have larger aggregates of liposomes and polyanionic active agent (nucleic acids), which may form during the production process, dissipate in such a way that polyanionic active agent (nucleic acid)-loaded amphoteric liposomes are ultimately present in the solution along with free, non-entrapped polyanionic active agent (nucleic acid) molecules.

Surprisingly, it was found that diluting the alcohol (ethanol) concentration and neutralizing the solution can be effected simultaneously, i.e., by adding a single neutralizing solution in an appropriate amount, with no substantial loss of entrapped active substance occurring and yields at comparable levels being achieved.

In a preferred embodiment of the invention following the formation of the liposomes the pH is increased to a pH which is at least one unit higher than the isoelectric point of the amphoteric liposomes, preferably the pH is increased to a pH>7. As mentioned above those skilled in the art are well known which pH value will result upon mixing two solutions of defined composition and in which way a solution must be modified in order to obtain the desired pH value of the mixture. For the adjustment of the pH value, buffers or bases well-known in the art can be used, including but are not limited to Tris-hydroxymethylaminomethan, BIS-TRIS, Carbonate, Triethanolamine, Triethylamine, Arginine, L-Arginine, Imidazole, hydrogenphosphate, HEPES or NaOH. Pharmaceutically acceptable buffers such as phosphate buffer are preferred.

In another embodiment of the invention the interactions can also be dissipated by increasing the ionic strength in the solution. Without being limited thereto, sodium chloride is preferably used to this end. Similarly, this method was found to dissipate the aggregates formed during the process, although the pH value remained constant under these conditions. The level of ionic strength required to prevent the interactions between the amphoteric liposomes and the polyanionic active agent depends on the lipid mixtures and polyanionic active agent being used. For example, large nucleic acids such as DNA plasmids bind more strongly to the liposomal membrane because larger numbers of repeating charges result in stronger binding. The final NaCl concentration used to dissipate the interactions between amphoteric liposomes and oligonucleotides is preferably between 250 and 1500 mM.

It will be appreciated that salts other than NaCl can also be used to increase the ionic strength, including but are not limited to sodium or potassium citrate, sodium or potassium phosphate, ammonium sulfate, or other salts. Sodium salts and of course pharmaceutically acceptable salts are particularly preferred. An increase of the ionic strength is also achieved by adding large amounts of buffer substance.

If the interactions are dissipated by increasing the ionic strength of the solution a further step including a pH increase and/or a dilution may follow.

Non-entrapped active substance can be removed in another step of the process. Methods well-known to those skilled in the art, such as gel filtration, centrifugation, dialysis or ultrafiltration, are suitable to this end. Moreover, the loaded amphoteric liposomes can be concentrated, if required. Similarly, methods well-known to those skilled in the art, such as centrifugation or ultrafiltration, are suitable to this end. Furthermore, these methods can be used to replace the aqueous medium or remove the water-miscible solvent.

The aqueous medium can be replaced by buffers including but are not limited to Tris-hydroxymethylaminomethan, BIS-TRIS, Carbonate, Triethanolamine, Triethylamine, Arginine, L-Arginine, Imidazole, hydrogenphosphate or HEPES. Pharmaceutically acceptable buffers such as phosphate buffer or Tris-hydroxymethylaminomethan are preferred.

As mentioned before, the active substance-containing liposomes prepared according to the method of the invention can be furthermore subjected to sterile filtration in a final step.

In one embodiment of the invention, an extrusion step can be performed between the process steps c) and d) and/or between d) and e) and/or between e) and f) and/or between f) and g), thereby allowing further improvement in size distribution of the active substance-containing liposomes.

In another embodiment of the invention the extrusion is performed at acid pH value, i.e., between the process steps c) and d) or d) and e), the amphoteric liposomes being or having formed being present in a state of cationic charge and interacting with the polyanionic active agent. For this reason, there is no or only slight release of previously entrapped polyanionic active agent.

Surprisingly, it was found that extrusion subsequent to dissipation of the interactions between the amphoteric liposomes having formed and the polyanionic active agent, i.e., between the process steps d) and e) and/or between e) and f) and/or between f) and g), neither results in a significant release of the entrapped polyanionic active agent. In another embodiment of the invention the active substance-containing amphoteric liposomes are extruded between the process steps d) and e) and/or between e) and f) and/or between f) and g), in which case preferably no more than 30% of entrapped active substance, more preferably no more than 20% of entrapped active substance, and especially preferably no more than 10% of entrapped active substance is released. Furthermore, it was surprising to find that the use of extrusion membranes with a pore size greater than the mean size of the active substance-containing amphoteric liposomes results in a reduced release of entrapped polyanionic active agent at a pH value of >7.

The loaded amphoteric liposomes can be extruded once or several times. In the case the liposomes are extruded several times one set of extrusion membranes may be used repeatedly or different sets of extrusion membranes may be used for each extrusion step. A set of extrusion membranes comprises one or more membranes placed in an extrusion device. If an apparatus as described below is used to prepare the loaded amphoteric liposomes with the process according to the present invention one or more extrusion devices can be included within the apparatus. Alternatively, the extrusion step(s) can be performed in an independent apparatus.

In another embodiment of the invention the active substance-containing amphoteric liposomes can be subjected to one or more freeze-thaw cycles between the process steps e) and f) and/or between f) and g), in which case it was surprising to find that this process results in a narrowed size distribution of the liposomes, with no release of significant amounts of entrapped active substance. In a preferred fashion the freeze-thaw cycles are effected in the presence of cryoprotectants, in which case sugars are preferably used. Pharmaceutically acceptable sugars are well-known to those skilled in the art and comprise e.g. glucose, fructose, sucrose, maltose or trehalose, without being limited thereto. Of course, alternative cryoprotectants can be used. Most are known in the art and include but are not limited to sugar alcohols like sorbitol or inositol, Trisaccarides like raffinose, Polysaccarides like ficoll or dextran and other polymers like polyvinylpyrrolidone or polyethylenglycol. In one embodiment the active substance-containing amphoteric liposomes having formed are added with said cryoprotectants prior to the freeze-thaw process. In a preferred embodiment the active substance-containing amphoteric liposomes are produced in the presence of cryoprotectants, so that the cryoprotectants are located within and outside the liposomes following production thereof. The amount of said cryoprotectants is preferably between 1 and 25%, more preferably between 5 and 15%. Freezing of the liposomes can be effected in liquid nitrogen, on dry ice, at −70° C. or −20° C., but is not limited to these methods.

Moreover, it was surprising to find that particular salts can increase the stability of the amphoteric liposomes. Inter alis, the cations of these salts contribute to such type of stabilization. Thus, the non-release of entrapped cargo during a freeze-thaw cycle can be improved by using tris(hydroxymethyl)aminomethane. Other suitable cations are triethanolamine, morpholine, piperazine, arginine or Larginine for example. Tris(hydroxymethyl)aminomethane and arginine are preferred cations. When using these cations, release of active substance less than possible with, e.g., sodium or potassium as cation is observed.

In some embodiments sodium may be a preferred cation, that can stabilize the amphoteric liposomes as well, whereas potassium as cation is not preferred.

In one embodiment, these cations are used as the only cations in the buffer system, i.e., a mixture of e.g. sodium ions with tris(hydroxymethyl)aminomethane ions is less preferred.

In another aspect of the use of the above preferred cations it was observed that the size of the liposomes produced is subject to less fluctuations after a freeze-thaw cycle than those when not using the preferred cations.

In another aspect of the use of preferred cations, improved colloid stability was observed. Without changing their colloidal properties such as size or viscosity, liposomes prepared using the preferred cations can be concentrated to higher levels than those when not using these cations. By using the preferred cations, it is possible e.g. to increase the concentration of the liposomes in a suspension to more than 100 mM, and in many cases the concentration can be increased to more than 130 mM, and in some cases, concentrating to 150 mM or more lipid is possible.

Surprisingly, it was found that the positive effects of the preferred cations and of the above-mentioned cryoprotectants show a synergistic behavior. As a result, a further improvement of process control or storage of the liposomes produced can be achieved. For example, such improvements comprise more accurate maintenance of the particle size, so that the difference in their mean size prior to and after a freeze-thaw cycle is less than 20%. Frequently, the synergistic use of cryoprotectants and cations allows to achieve a size difference of less than 10% of the mean size, and in many cases the difference in particle size is below a reliably detectable difference.

Likewise, low-level release of entrapped active substance or cargo is advantageous, and such release can be reduced to values of less than 10% by means of the measures described above; in many cases, a release of less than 5% is possible, and a release of less than 2% of entrapped active substance is frequently achieved.

The preferred cations of the buffer system and the cryoprotectants can be combined at will. Thus, for example, combinations of sucrose and tris(hydroxymethyl)aminomethane as cation are possible, but also those of maltose or trehalose.

In a preferred embodiment of this aspect of the invention, the amount of cryoprotectants employed can be reduced by adding the preferred cations, without substantially influencing the release of active substance or the size of the particles.

In another embodiment of the invention the active substance-containing amphoteric liposomes, following the process step e) or f) or g), are likewise subjected to one or more freeze-thaw cycles and/or to lyophilization. Surprisingly, as described above, it was found that the active substance-containing liposomes produced according to the method of the invention can be frozen preferably in the presence of cryoprotectants and optionally of the above-mentioned salts, and following thawing, a narrowed size distribution of the liposomes is usually observed, with no release of significant amounts of entrapped active substance. For this reason, the active substance-containing liposomes produced according to the method of the invention are frozen after the final process step, which can be e), f) or g), and stored in frozen condition in a preferred embodiment of the invention. In another embodiment of the invention the active substance-containing liposomes produced according to the method of the invention can be lyophilized after the final process step which can be e), f) or g). Similarly, lyophilization is preferably effected with addition of the above-explained cryoprotectants and salts. Thereafter, the liposomes can be stored e.g. at room temperature.

Another important parameter is the process temperature. For liposome formation, it is invariably selected higher than the phase transition temperature of the lipid mixture. The phase transition temperature can be determined using calorimetric procedures, e.g. DSC (differential scanning calorimetry). The lipid having the highest phase transition temperature as a pure substance can be used for a rough determination of the process temperature. To ensure a safety gap, the process temperature should be selected to be at least 5° C. above the determined or approximated phase transition temperature of the lipid mixture.

The production of the polyanionic active agentcontaining amphoteric liposomes can be effected without any further devices, e.g. by mixing in a stirred container or in the devices described above.

In a preferred embodiment the method according to the invention can also be conducted as a continuous process, e.g. in the following apparatus.

FIG. 1 shows the basic design of the apparatus. Via tubes and/or pipe connections L1 and L2 made of chemically inert material and using pumps P1 and P2 (preferably operating at lowest possible pulse), the components to be mixed are conveyed from temperature-controlled reservoirs G1 and G2 holding the alcoholic lipid solution and the aqueous nucleic acid (or other polyanionic active agent) solution to the mixing chamber M1.

The mixing chamber consists of a V mixer which has the advantage of a very small mixing volume. In addition, other mixer designs are also possible, such as T mixers, micromixers, dynamic or static/dynamic mixers.

The overall stream after leaving the mixing chamber M1 is constituted of the sum of the separate streams.

The overall stream is passed through another tube and/or pipe connection L3 into a receiver G3 which holds the buffer for dilution and/or adjustment of the detaching conditions. The tube or pipe connection L3 may serve as a timer wherein the liposomes having formed can be held under binding conditions for a specific time until binding of active substance to the lipid membrane has reached its maximum value. The residence time is determined by the tube volume and the overall flow rate.

Timer L3 and receiver G3 can also be temperature-controlled.

The buffer for dilution and/or adjustment of the detaching conditions can also be supplied from the vessel G3 via a tube and/or pipe connection via another mixing chamber M2 by means of pump P3, and the overall stream is collected in another vessel G4 after passing the mixing chamber M2. The mixing chamber M2 may have the same design as M1. This embodiment is illustrated in FIG. 2.

In case when the dilution and adjustment step is separately performed, another set of tube and/or pipe connection, mixing chamber M3, pump P4 and vessel G5 may be implemented as illustrated in FIG. 11. Alternatively the stream from mixing chamber M2 can be directly injected into vessel G5 containing the dilution or adjustment buffer.

Moreover, the separate stream of the aqueous solution of active substance can be formed of two individual separate streams prior to feeding into the system. In particular, this is advantageous in those cases where the solution of active substance is present in the form of a concentrate and must be suitably diluted with the lipid solution prior to mixing or when the solution of active substance must be re-buffered prior to feeding. This is particularly advantageous in those cases where the dissolved active substance is sensitive to the binding conditions and cannot be stored for a long time under such conditions. Referring to FIG. 3, this embodiment requires the use of vessel G1a, pump P1a and mixing chamber M1a, together with the corresponding pipe connections. Of course, also the lipid solution may be acidified just prior to the mixing. Accordingly, as illustrated in FIG. 12, this embodiment requires the use of vessel G2a, pump P2a and mixing chamber M2a, together with the corresponding pipe connections.

Furthermore one or more sterile filters may be included in the apparatus. In one embodiment of the invention the one or more sterile filters are placed between pump 1 (P1) and/or pump 2 (P2) and/or pump 3 (P3) and/or pump 1a (P1a) and/or pump 2a (P2a) and/or pump 4 (P4) and the respective mixing chambers M1 and/or M2 and/or M1a and/or M2a and/or M3. This performance is in particular advantageous if an aseptic process is required. In addition or alternatively one or more sterile filters may be placed after the final mixing chamber. FIG. 13 illustrates exemplarily this embodiment.

In addition or alternatively one or more extrusion membrane holders fitted with one or more extrusion membranes may be placed between the mixing chambers M1 and M2 and/or M2 and M3 and/or downstream M3 as illustrated exemplarily in FIG. 14.

The quality characteristics of the liposomes produced, such as size, size distribution and active substance content can be controlled reproducibly by means of a suitable process design of the process parameters. The process parameters which can be varied comprise: amount of ethanol, lipid concentration, nucleic acid (or other polyanionic active agent) concentration, N/P ratio, composition of the aqueous nucleic acid solution, composition of the alcoholic lipid solution, temperature, volume flows and pressure.

Another object of the invention is to provide process parameters allowing to control the size of the amphoteric liposomes and the active substance/lipid ratio in the final product.

In contrast to “passive” inclusion of active substances in liposomes, it was surprising to find that the inclusion efficiency in the method according to the invention is increased at lower lipid concentrations in the process. However, the lipid concentration in the process also influences the size of the liposomes, which initially increases with increasing lipid concentration, to reach a constant value. Preferred lipid operating concentrations in the method according to the invention are between 0.2 and 10 mM lipid, more preferably between 0.5 and 5 mM lipid, and most preferably between 0.5 and 3 mM, wherein said lipid concentrations base on a lipid concentration at process step (e), i.e. after the optional dilution step (d). In some embodiments lipid operating concentrations up to 25 mM may be preferably as well, for example in cases in which a reduction of the volume of solutions in the process is desirable.

In addition it was found that the amount of the water-miscible solvent in the mixture may have an impact on the encapsulation efficiency of the nucleic acid active substance. The use of higher amounts of water-miscible solvents in the mixture (e.g. 30% by volume) combined with a subsequent dilution step can lead to higher encapsulation efficiencies in some embodiments.

Moreover, and quite surprisingly, a temperature dependence of the inclusion efficiency was found in the method according to the invention. Thus, higher inclusion efficiency is achieved at elevated temperatures. The preferred temperature range for the production of nucleic acid-loaded amphoteric liposomes by means of the method according to the invention is between 20 and 50° C.

Furthermore, increasing the ionic strength or osmolarity in the aqueous solution of active substance affords surprising results in the method according to the invention. The inclusion efficiency decreases up to a specific ionic strength, while at the same time there is an increase in size of the liposomes being formed. Initially, the size of the liposomes and the inclusion efficiency remain unchanged when further increasing the ionic strength. Ultimately, the interaction between the amphoteric liposomes and the nucleic acids is completely suppressed at very high ionic strength, so that inclusion proceeds via the “passive” process. This is reflected in the smaller size of the amphoteric liposomes being formed. Without being limited thereto, the ionic strength can be increased by addition of sodium chloride. Of course, other suitable pharmaceutically acceptable salts capable of changing the ionic strength of a solution are well-known to those skilled in the art. These include e.g. citrate, phosphate or acetate, without being limited thereto. In a preferred fashion the method according to the invention is carried out using an ionic strength between 0 and 250 mM, and preferably between 0 and 150 mM. In a specific embodiment the method is performed at an ionic strength between 0 and 50 mM.

In contrast, a different effect appears when adding non-ionic ingredients, e.g. a sugar (sucrose) instead of salt to the aqueous solution of active substance in order to increase the osmolarity. Surprisingly, the size of the nucleic acid-loaded amphoteric liposomes produced by the method according to the invention remained nearly constant, while the inclusion efficiency increased. In a preferred fashion the method according to the invention is performed using a sugar concentration between 0 and 500 mM. Pharmaceutically acceptable sugars are well-known to those skilled in the art and include but are not limited to e.g. sucrose, glucose or trehalose.

The variation of the initial molar ratio of cationic charges (N) of the lipids to anionic charges (P) of the nucleic acid or other polyanionic active agent (N/P ratio) has also an impact on the formation of nucleic acid loaded amphoteric liposomes. It was found that the higher the initial N/P ratio the lower the size of the resulting drug loaded amphoteric liposomes, whereas the encapsulation efficiency remains unaffected. It should be emphasized that the selection of the initial N/P ratio influences not only the size of the liposomes but also the final drug/lipid ratio of the loaded amphoteric liposomes. For example, if a low final drug/lipid ratio is desired high initial N/P ratios are required. Conversely, if a product should have a high final drug/lipid ratio the process should be started with a lower initial N/P ratio.

In addition, the velocity of mixing the water-miscible lipid solution and the aqueous nucleic acid solution is also a process parameter that can be used to control the size of the liposomes within the method of the present invention. It was found that a faster mixing of the solutions results in smaller sized nucleic acid loaded amphoteric liposomes.

Moreover, also the composition of buffers and the lipid solution may have an impact on the formation of nucleic acid loaded amphoteric liposomes according to the method of the present invention. Of course, changing the buffer composition often includes a change of the ionic strength and/or osmolarity and/or the ions (e.g. cations) in the system. The influence of these process parameters are discussed above.

By varying the process parameters of the invention, it is possible to produce loaded amphoteric liposomes of a desired specification. For pharmaceutical uses, the size of nucleic acid-loaded amphoteric liposomes is preferably between 50 nm and 500 nm, and more preferably between 80 nm and 250 nm. Furthermore, the liposomes produced by means of the method according to the invention can be uni-, oligo- or multilamellar. For oligonucleotide active substances, the active substance/lipid ratio preferred in the final product is preferably between 1 mg of oligonucleotide per g of lipid and 300 mg of oligonucleotide per g of lipid. More preferably, the active substance/lipid ratio is between 10 and 100 mg of oligonucleotide per gram of lipid. For larger nucleic acid active substances, such as DNA plasmids, the preferred active substance/lipid ratio is between 0.3 mg of DNA per g of lipid and 30 mg of DNA per g of lipid.

In one embodiment the nucleic acids are oligonucleotides. Preferably, the oligonucleotide loaded amphoteric liposomes prepared by the process according to the present invention are multilamellar.

Multilamellar as defined herein means that the liposomes comprise a plurality of lipid bilayers. In contrast, oligolamellar as defined herein means that the liposomes comprise a few lipid bilayers. Unilamellar as defined herein means that the liposomes comprise just one lipid bilayer.

In a preferred embodiment the oligonucleotide loaded amphoteric liposomes prepared by a process according to the present invention have a size of between 70 and 150 nm and a final oligonucleotide/lipid ratio of between 40 and 120 mg oligonucleotide per gram lipid.

Alternatively the size of the oligonucleotide loaded amphoteric liposomes prepared by a process according to the present invention is between 70 and 150 nm and the drug/lipid ratio between 1 and 40 mg oligonucleotide per gram lipid.

In another preferred embodiment the oligonucleotide loaded amphoteric liposomes prepared by a process according to the present invention have a size of between 130 and 200 nm and a final oligonucleotide/lipid ratio of between 1 and 40 mg oligonucleotide per gram lipid.

Alternatively, the oligonucleotide loaded amphoteric liposomes prepared by a process according to the present invention have a size of between 130 and 200 nm and a final oligonucleotide/lipid ratio of between 40 and 120 mg oligonucleotide per gram lipid.

In still another embodiment, the nucleic acids having a chain length of more than 50 nucleobases or basepairs, preferably more than 100 nucleobases or basepairs and include without limitation circular DNA plasmids, linear DNA constructs, like MIDGE vectors, RNAs, aptamers or ribozymes. Preferably, the size of liposomes loaded with such nucleic acids and prepared by the method according of the present invention is between 70 and 300 nm and the final nucleic acid/lipid ratio of said liposomes is between 0.3 and 30 mg of nucleic acid per g of lipid. Preferably, the nucleic acid loaded amphoteric liposomes of this embodiment are oligolamellar.

One aspect of the present invention relates to nucleic acid loaded amphoteric liposomes produced by the method according to the present invention.

In specific embodiments of this aspect nucleic acid loaded amphoteric liposomes with defined specifications like the size of the liposomes and the final drug/lipid ratio may be prepared. Following examples illustrate but do not limit the inventive use of specific process parameters to prepare oligonucleotide loaded amphoteric liposomes with defined specifications by the method according to the present invention. Of course, one skilled in the art can vary process parameters described within the present invention to prepare nucleic acid loaded amphoteric liposomes of alternative lipid compositions and specifications.

1. Lipid composition: POPC/DOPE/MoChol/Chems 6:24:47:23

Lipid solution: 25 mM lipid in isopropanol+10 mM HCl

Oligonucleotide solution: 541 μg/ml in 10 mM HAc, 300 mM Sucrose, pH adjusted to pH 4 with 2M Tris solution

Initial N/P ratio: 2

Mixing ratio: 1/2.33 (lipid/oligonucleotide)→30% alcohol

pH shift including dilution: 2 Volumes of 150 mM Tris, pH 7.5→10% alcohol

Size: 117 nm

Final drug/lipid ratio [mg/g]: 89.1

2. Lipid composition: POPC/DOPE/MoChol/Chems 6:24:47:23

Lipid solution: 50 mM lipid in isopropanol+10 mM HCl

Oligonucleotide solution: 361 μg/ml in 10 mM HAc, 300 mM Sucrose, pH adjusted to pH 4 with 2M Tris solution

Initial N/P ratio: 6

Mixing ratio: 1/2.33 (lipid/oligonucleotide)→30% alcohol

pH adjustment including dilution: 2 Volumes of 150 mM Tris, pH 7.5→10% alcohol

Size: 78 nm

Final drug/lipid ratio [mg/g]: 27.8

3. Lipid composition: POPC/DOPE/MoChol/DMG-Succ 6:24:23:47

Lipid solution: 10 mM lipid in isopropanol+10 mM HAc

Oligonucleotide solution: 53 μg/ml in 20 mM HAc, 300 mM Sucrose, pH adjusted to pH 4 with 2M Tris solution

Initial N/P ratio: 4

Mixing ratio: 1/2.33 (lipid/oligonucleotide)→30% alcohol

pH adjustment including dilution: 2 Volumes of 136 mM Na₂HPO₄, 100 mM NaCl→10% alcohol

Size: 193 nm

Final drug/lipid ratio [mg/g]: 11.3

4. Lipid composition: POPC/DOPE/MoChol/Chems 15:45:20:20

Lipid solution: 50 mM lipid in isopropanol+10 mM HAc

Oligonucleotide solution: 205 μg/ml in 20 mM HAc, 300 mM Sucrose, pH adjusted to pH 4 with 2M Tris solution

Initial N/P ratio: 4.5

Mixing ratio: 1/2.33 (lipid/oligonucleotide)→30% alcohol

pH adjustment including dilution: 2 Volumes of 136 mM Na₂HPO₄, 100 mM NaCl→10% alcohol

Size: 180 nm

Final drug/lipid ratio [mg/g]: 18.7

5. Lipid composition: POPC/DOTAP/Chems 25:28:47

Lipid solution: 25 mM lipid in isopropanol

Oligonucleotide solution: 575 μg/ml in 10 mM HAc, 300 mM Sucrose, pH adjusted to pH 4 with 2M Tris solution

Initial N/P ratio: 1.6

Mixing ratio: 1/2.33 (lipid/oligonucleotide)→30% alcohol

pH adjustment including dilution: 2 Volumes of 150 mM Tris, pH 7.5→10% alcohol

Size: 153.7 nm

Final drug/lipid ratio [mg/g]: 78.3

Following is a description by way of example only with reference to the accompanying drawings of embodiments of the present invention.

### DESCRIPTION OF THE FIGURES

FIG. 1 is a schematic representation of a device for the continuous operation of the method according to the invention.

FIG. 2 is a schematic representation of a device for the continuous operation of the method according to the invention, with admixing of buffer for dilution and/or adjustment of the detaching conditions.

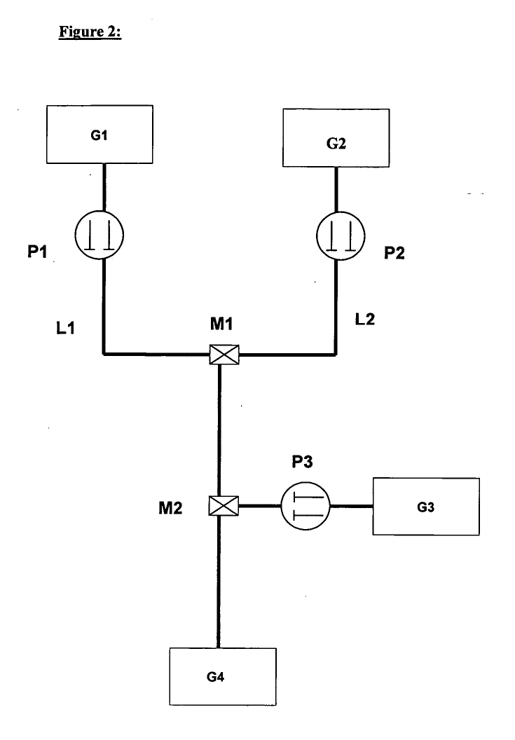

FIG. 3 is a schematic representation of a device for the continuous operation of the method according to the invention, with additional treatment of the aqueous solution of active substance by re-buffering or diluting.

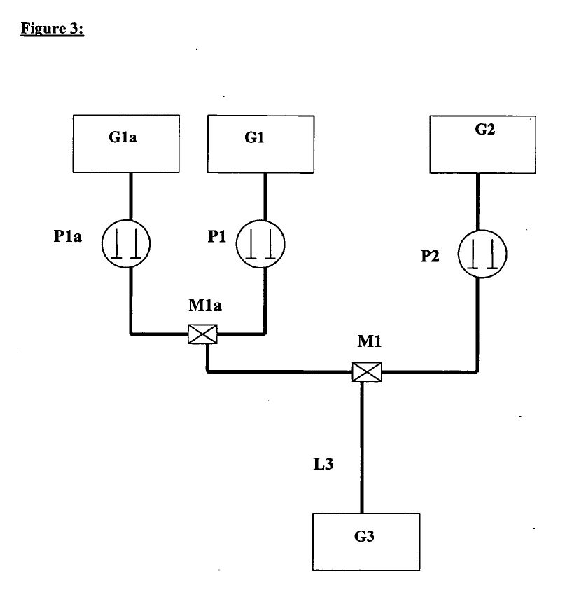

FIG. 4: Influence of the lipid concentration on the size of the resulting liposomes of Example 1.

FIG. 5: Influence of the lipid concentration on the inclusion efficiency of the resulting liposomes of Example 1.

FIG. 6: Influence of the temperature on the size of the resulting liposomes of Example 1.

FIG. 7: Influence of the temperature on the inclusion efficiency of the resulting liposomes of Example 1.

FIG. 8: Influence of the NaCl concentration on the size of the resulting liposomes of Example 1.

FIG. 9: Influence of the NaCl concentration on the inclusion efficiency of the resulting liposomes of Example 1.

FIG. 10: Influence of increasing amounts of ethanol on the permeability of amphoteric liposomes.

FIG. 11 is a schematic representation of a device for the continuous operation of the method according to the invention, with admixing of buffer for dilution and separately adjustment of the detaching conditions.

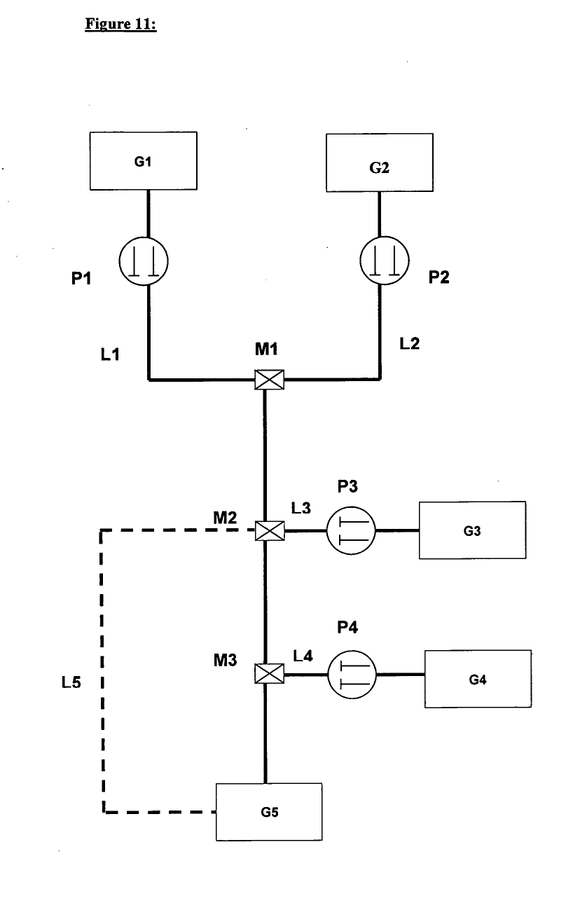

FIG. 12 is a schematic representation of a device for the continuous operation of the method according to the invention, with additional treatment of the alcoholic solution of lipids by re-buffering or diluting.

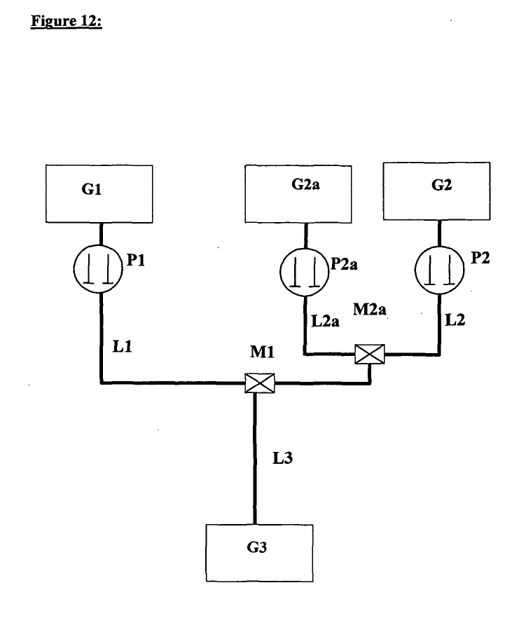

FIG. 13 is a schematic and exemplarily representation of a device for the continuous operation of the method according to the invention, including sterile filters between the pumps and the mixing chambers and after the final mixing chamber.

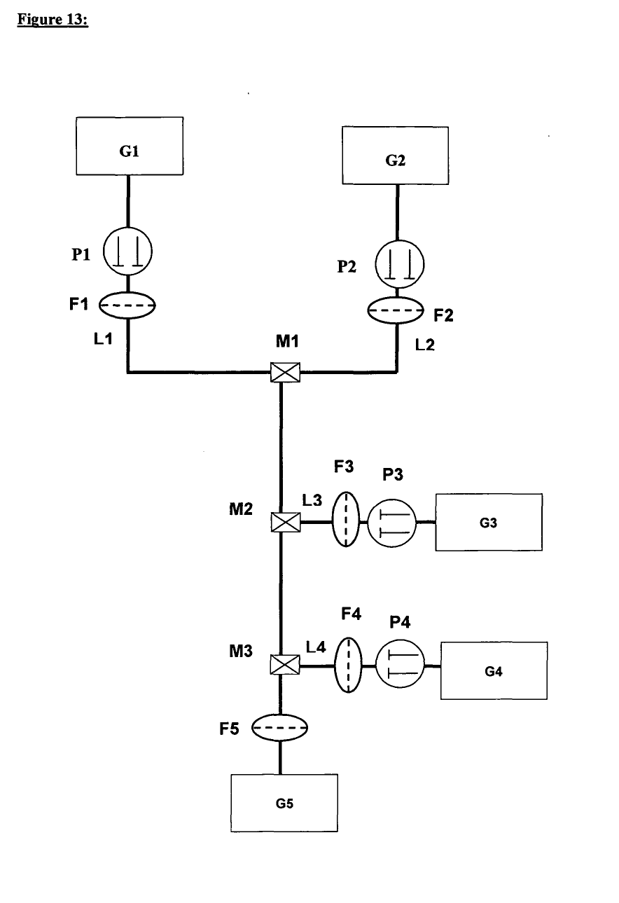

FIG. 14 is a schematic and exemplarily representation of a device for the continuous operation of the method according to the invention, including extrusion membrane holders fitted with one or more extrusion membranes between the mixing chambers M1 and M2 and/or M2 and M3 and/or down-stream M3.

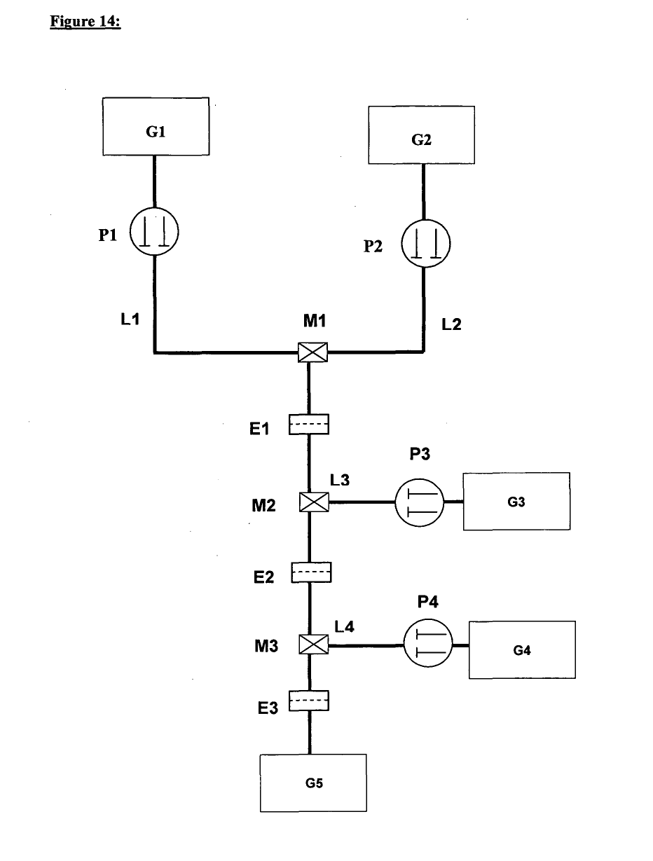

### EXAMPLE 1

### Production of Antisense Oligonucleotide-Loaded Amphoteric Liposomes

Variation of Various Process Parameters

In this experiment, an 18mer antisense oligonucleotide was entrapped in amphoteric liposomes having the following composition:

| | |
|---|---|
| POPC/DOPE/MoChol/Chems | 15:45:20:20 |

The following process parameters were varied consecutively:

Lipid concentration

Temperature

NaCl concentration in the aqueous antisense solution

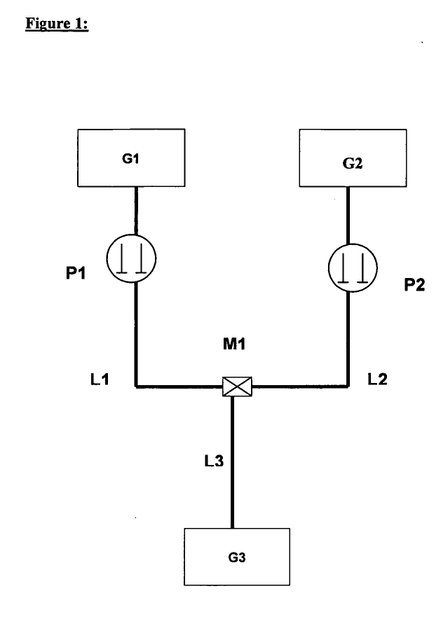

The production of the antisense-loaded amphoteric liposomes was effected using the apparatus represented in FIG. 1.

To combine the two metered separate streams of provided lipid solution and antisense solution, the following volume flows were selected:

Volume flow, lipid: 10 ml/min

Volume flow, antisense: 90 ml/min

The batch size produced was 40 ml each time.

To dissipate the interactions between the amphoteric liposomes and the antisense molecules following formation of the liposomes, 1/20 volume of 1M Tris solution, pH 8, was added with stirring.

Variation of Lipid Concentration

The following process parameters were kept constant during production:

TABLE 2

| | |
|---|---|
| N/P | 2.37 |
| Amount of ethanol | 10% |
| Temperature | Room temperature |
| Composition of aqueous | 20 mM acetate, |
| antisense solution | 50 mM NaCl, |
| | pH 4.5 |

To keep the N/P ratio constant, the concentration of the antisense solution was adapted to the corresponding lipid concentration.

TABLE 3

| Lipid stock | Final lipid | |
|---|---|---|
| solution | concentration | Final CD40 |
| [mM] | [mM] | [μg/ml] |
| 30 | 3 | 90 |
| 20 | 2 | 60 |
| 15 | 1.5 | 45 |
| 10 | 1 | 30 |
| 5 | 0.5 | 15 |

Variation of Temperature

The following process parameters were kept constant during production:

TABLE 4

| | |
|---|---|
| N/P | 2.37 |
| Amount of ethanol | 10% |
| Final lipid concentration | 0.5 mM |
| Composition of aqueous | 20 mM acetate, |
| antisense solution | 50 mM NaCl, |
| | pH 4.5 |

The temperature was varied as follows: 20° C., 30° C., 40° C., 50° C.

Variation of the NaCl Concentration in the Aqueous Antisense Solution

The following process parameters were kept constant during production:

TABLE 5

| | |
|---|---|
| N/P | 2.37 |
| Amount of ethanol | 10% |
| Temperature | Room temperature |
| Final lipid concentration | 0.5 mM |
| Buffer and pH of the | 20 mM acetate, |
| aqueous antisense solution | pH 4.5 |

The NaCl concentration of the aqueous antisense solution was successively increased in a range of from 0 mM to 400 mM. The buffer composition and pH value remained unchanged.

Determination of the Liposomes Size:

Following dilution of the samples (max. 1% ethanol) in PBS, the size of the liposomes was determined using photon correlation spectroscopy (PCS).

Removal of Non-Entrapped Antisense Oligonucleotide:

Non-entrapped antisense oligonucleotide was removed by sedimentation of the liposomes.

Determination of the Inclusion Efficiency of the Antisense Oligonucleotides in Amphoteric Liposomes:

The liposomes were dissolved in a solvent mixture (chloroform and methanol=1+4), and the amount of entrapped antisense was determined using OD measurement at 260 nm.

Determination of the Lipid Concentration:

Following extraction into chloroform, the lipid was determined as inorganic phosphate (P. Veldhofen and G. Mannaerts (1987) Anal. Biochem. 161, 45-48).

Results:

Variation of the Lipid Concentration

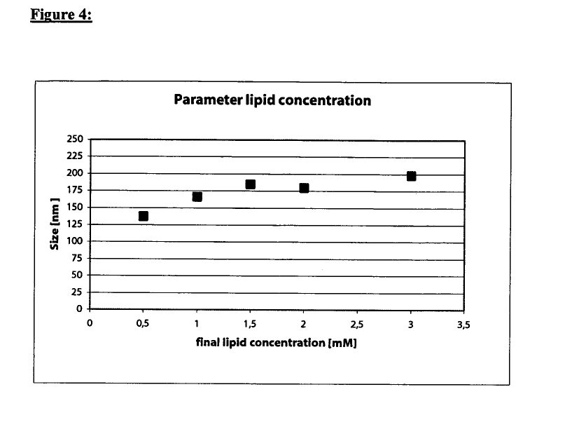

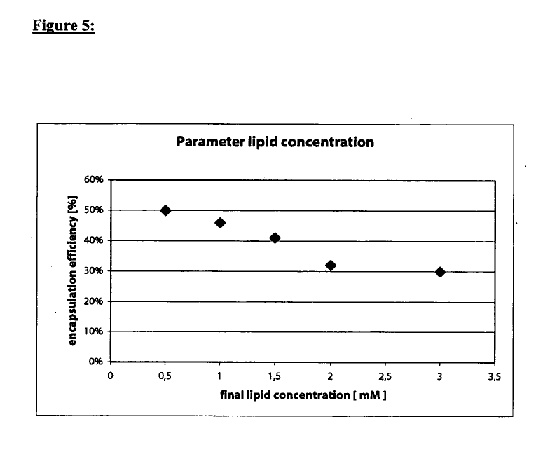

FIG. 4 shows that the size of the liposomes initially increases with increasing lipid concentration, to become stable from a final lipid concentration of 1.5 mM on. FIG. 5 illustrates the inclusion efficiency of the antisense oligonucleotides in dependence of the lipid concentration. The inclusion efficiency decreases with increasing lipid concentration.

Variation of the Temperature

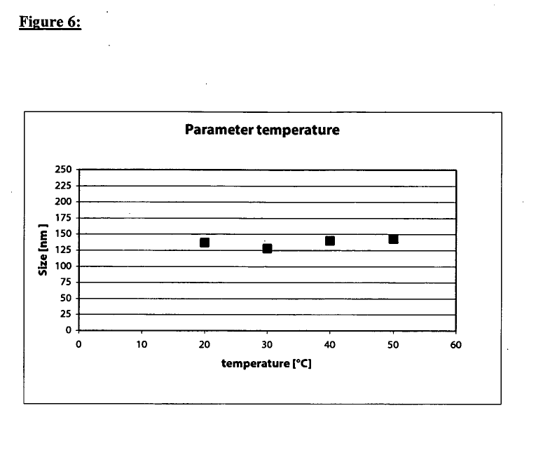

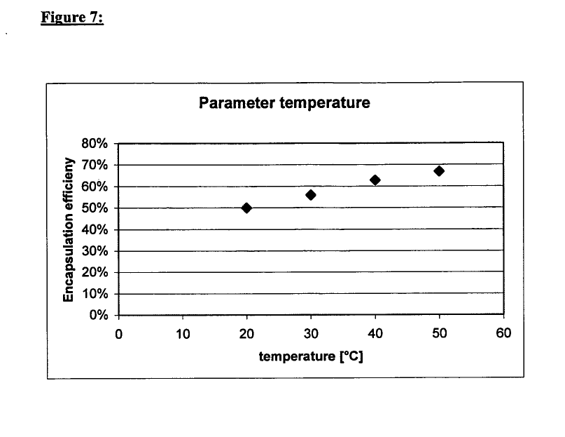

The size of the liposomes is not changed with increasing temperature, as shown in FIG. 6. In contrast, FIG. 7 shows that improved inclusion efficiencies are achieved with increasing temperature.

Variation of the NaCl Concentration in the Aqueous Antisense Solution

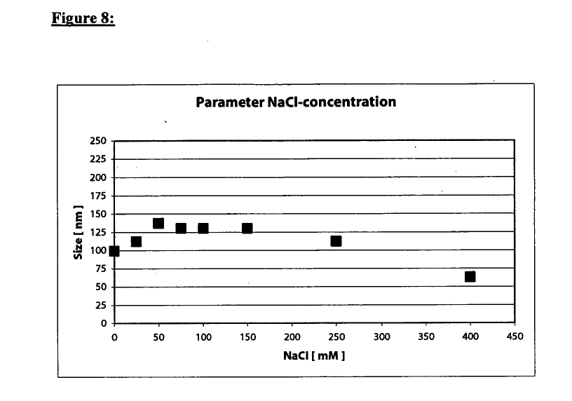

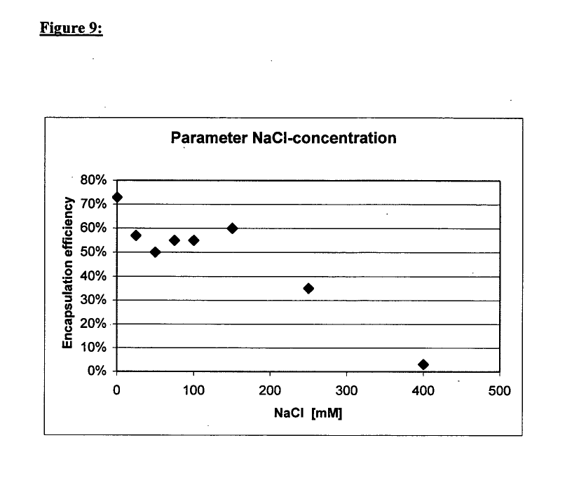

FIG. 8 shows that the size of the liposomes initially increases slightly with increasing NaCl concentration in the aqueous antisense oligonucleotide solution, but subsequently remains relatively constant over a wide range. The inclusion efficiency is best without addition of NaCl, as demonstrated in FIG. 9. The inclusion efficiency initially decreases by about 20% when adding NaCl, but subsequently remains relatively constant up to an NaCl concentration of about 125 mM. It is only at even higher salt concentrations that the inclusion efficiency decreases significantly. At a salt concentration of 400 mM, virtually no antisense oligonucleotide is entrapped in the amphoteric liposomes anymore.

### Example 2

### Treatment of Various Amphoteric Liposomes with Increasing Amounts of Ethanol

Preparation of Various Amphoteric Liposomes with Entrapped Carboxyfluorescein Fluorescent Dye

Using lipid stock solutions in chloroform, various lipid mixtures were mixed and the solvent was removed in a rotary evaporator. The resulting lipid films were dried overnight in vacuum. Thereafter, the lipid films were hydrated with 100 mM carboxyfluorescein (CF) in PBS, pH 7.5. The resulting lipid concentration was 20 mM. The suspensions were hydrated for 20 min at RT, homogenized for 5 min in an ultrasonic bath, and finally subjected to 3 freeze-thaw cycles. Following final thawing, the liposomal suspensions were extruded 15 times through 100 nm polycarbonate membranes. Non-entrapped CF was removed by gel filtration, so that the liposomes were diluted by a factor of 3.

The following formulations were produced:

| | |
|---|---|
| Form. 1 POPC/DOPE/MoChol/Chems | 6:24:47:23 |
| Form. 2 POPC/DOPE/MoChol/DMG-Succ | 6:24:35:35 |
| Form. 3 POPC/DOPE/MoChol/Chems | 15:45:20:20 |
| Form. 4 POPC/DOPE/MoChol/Chems | 10:30:30:30 |
| Form. 5 POPC/DOPE/MoChol/DMGSucc | 26:34:20:20 |

Influence of Increasing Amounts of Ethanol on the Integrity of the Liposomes

Initially, the liposomes were diluted by a factor of 32 with PBS, and 20 μl of this dilution was added with 180 μl of a PBS/ethanol mixture with increasing amounts of ethanol in a black microtiter plate and incubated for 30 min. Subsequently, the CF released from the liposomes was measured using fluorescence measurement at 490 nm/530 nm. To obtain a reference value for complete release, the batches were added with 20 μl of 2.5% Triton X-100 solution and the fluorescence was measured again.

Result

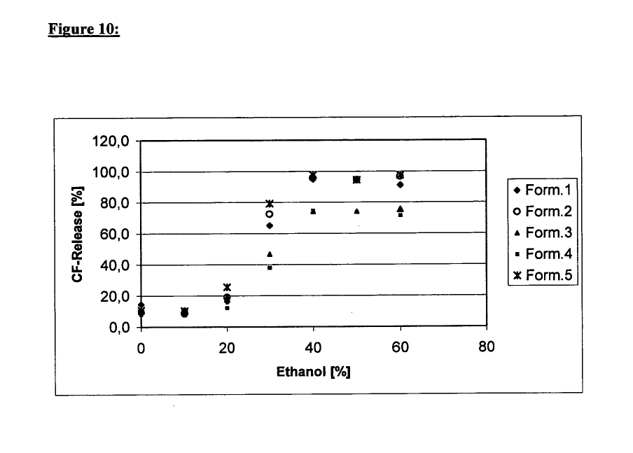

FIG. 10 illustrates the release of the carboxyfluorescein fluorescent dye from the amphoteric liposomes with increasing amounts of ethanol added from outside. From a critical ethanol concentration of about 30% ethanol on, the liposomes become permeable and release the entrapped dye.

### Example 3

### Preparation of Various Nucleic Acid-Loaded Amphoteric Liposomes Using 10% or 30% Ethanol Injection

The lipid mixtures were weighed and dissolved in ethanol p.a., so as to make a lipid concentration of 40 mM or 13.3 mM. 0.5 ml and 1.5 ml, respectively, of these ethanolic lipid solutions were injected into 4.5 ml and 3.5 ml, respectively, of an aqueous nucleic acid solution (18mer antisense oligonucleotide) in 90 mM acetate, 300 mM sucrose, pH 4, with stirring. The amount of antisense oligonucleotide was calculated such that an N/P ratio of 3 was obtained in the batch. Following preparation, each liposome suspension was diluted with 10 ml of 120 mM Na₂HPO₄, 90 mM NaCl, pH 9, so as to obtain a pH value of >7. To determine the inclusion efficiency, non-entrapped antisense oligonucleotide was removed using Centriprep ultrafiltration units (100 kDa MWCO) and determined using OD measurement at 260 nm. Following dilution of the samples (max. 1% ethanol) in PBS, the size of the liposomes was determined using photon correlation spectroscopy (PCS).

The following formulations were produced as described:

TABLE 6

| | | | | Lipid |
|---|---|---|---|---|
| Formulation | Composition | % EtOH | N/P | conc. |
| 1 | MoChol/Chems | 10 | 3 | 4 mM |
| | 67:23 | | | |
| 2 | MoChol/Chems | 30 | 3 | 4 mM |
| | 67:23 | | | |
| 3 | POPC/DOPE/MoChol/Chems | 10 | 3 | 4 mM |
| | 6:24:47:23 | | | |
| 4 | POPC/DOPE/MoChol/Chems | 30 | 3 | 4 mM |
| | 6:24:47:23 | | | |
| 5 | POPC/DOTAP/Chems | 10 | 3 | 4 mM |
| | 25:28:47 | | | |
| 6 | POPC/DOTAP/Chems | 30 | 3 | 4 mM |
| | 25:28:47 | | | |
| 7 | POPC/DOPE/MoChol/Chems | 10 | 3 | 4 mM |
| | 15:45:20:20 | | | |
| 8 | POPC/DOPE/MoChol/Chems | 30 | 3 | 4 mM |
| | 15:45:20:20 | | | |

Result:

The following inclusion efficiencies and liposome sizes were obtained at the end of the preparation:

TABLE 7

| | | % inclusion | |
|---|---|---|---|
| Formulation | Composition | efficiency | Size in nm |
| 1 | MoChol/Chems | 21 | 102 |
| | 67:23 | | |
| 2 | MoChol/Chems | 55 | 123 |
| | 67:23 | | |
| 3 | POPC/DOPE/MoChol/Chems | 70 | 146 |
| | 6:24:47:23 | | |
| 4 | POPC/DOPE/MoChol/Chems | 84 | 124 |
| | 6:24:47:23 | | |
| 5 | POPC/DOTAP/Chems | 80 | 178 |
| | 25:28:47 | | |
| 6 | POPC/DOTAP/Chems | 75 | 121 |
| | 25:28:47 | | |
| 7 | POPC/DOPE/MoChol/Chems | 41 | 168 |
| | 15:45:20:20 | | |
| 8 | POPC/DOPE/MoChol/Chems | 68 | 199 |
| | 15:45:20:20 | | |

### Example 4

### Preparation of Nucleic Acid-Loaded Amphoteric Liposomes Using the Apparatus Illustrated in FIG. 1

The lipid mixtures were weighed and dissolved in ethanol p.a. to make a lipid concentration of 16.6 mM. The 18mer antisense oligonucleotide was dissolved in 20 mM Na acetate, 300 mM sucrose, pH 4.0. The amount of antisense oligonucleotide was calculated so as to obtain an N/P ratio 3 or 4 in the batch (see below). The two solutions were mixed at flow rates of 24 ml/min (lipid solution) and 56 ml/min (antisense solution), respectively, in a device in accordance with FIG. 1. Following preparation, the liposome suspensions were diluted to a 10% ethanol content (20 mM Na acetate, 300 mM sucrose, pH 4.0). Thereafter, the suspensions were rebuffered to pH 7.5 using ⅕ volume of 1 M Tris, pH 8. Non-entrapped oligonucleotides and ethanol were removed by crossflow dialysis, and the liposomes were subsequently concentrated.

The liposomes were dissolved in a solvent mixture (chloroform and methanol=1+4), and the amount of entrapped antisense was determined using OD measurement at 260 nm.

Determination of the Lipid Concentration:

Following extraction into chloroform, the lipid was determined as inorganic phosphate (P. Veldhofen and G. Mannaerts (1987) Anal. Biochem. 161, 45-48).

The size of the liposomes was determined in PBS using photon correlation spectroscopy (PCS).

Result:

TABLE 8

| | | POPC/DOPE/ | POPC/DOPE/ | |
|---|---|---|---|---|
| | | Mochol/Chems | Mochol/Chems | MoChol/Chems |
| | Formulation | 15:45:20:20 | 6:24:47:23 | 67:23 |
| Input | N/P | 3 | 3 | 4 |
| | Process | 1085 | 876 | 835 |
| | volume | | | |
| | (ml) | | | |
| | Lipid conc. | 5 | 5 | 5 |
| | (mM) | | | |
| | ASO | 0.112 | 0.264 | 0.282 |
| | (mg/ml) | | | |
| Output | Lipid conc. | 158 | 75 | n.d. |
| | (mM) | | | |
| | ASO | 2.21 | 3.81 | 3.96 |
| | (mg/ml) | | | |
| Result | Inclusion | 62% | 96% | n.d. |
| | efficiency | | | |
| | Size (nm) | 112 | 109 | 112 |
| | PI | 0.19 | 0.28 | 0.26 |

### Example 5

### Influence of Various Parameters on the Freeze-Thaw Ability

The liposomes as described in Example 4 were produced with the following parameters: 80 mM ethanolic lipid solution consisting of 6 mole-% POPC, 24 mole-% DOPE, 23 mole-% MoChol and 47 mole-% DMGSucc, 25% ethanol injection, N/P=2. Dilution to 15% ethanol and rebuffering to pH 7.5 were effected in a single step with a flow rate of 25 ml/min, using a solution consisting of 500 mM sodium hydrogen phosphate, pH 9. The diluted suspension was concentrated to 160 mM lipid by means of crossflow filtration, whereby non-entrapped CD40 oligonucleotide and ethanol were simultaneously removed.

The liposome preparation was aliquoted and examined for its freeze-thaw behavior according to the parameters in the following table. Unless otherwise specified, the samples were subsequently frozen at −70° C. and thawed at room temperature. The treated liposomes were tested for particle size and particle size distribution (as in Example 4). The amount of released active substance was separated using ultrafiltration (Centrisart, 100 kD, 2500×g, 2 hours, 20° C.) and determined by means of OD_{260 nm} against a reference solution including CD40.

TABLE 9

| Lipid concentration | Sugar | Additives/ | ml | | Size in | Poly | Released active |
|---|---|---|---|---|---|---|---|
| in mM | concentration | actions | Sample | Production/mixture | nm | index | substance/notes |
| 160 | 10% sucrose | — | 1 | — | 281 | 0.165 | Gel |
| 140 | 10% sucrose | — | 0.875 | ad 1 ml PBsucrose | 249 | 0.099 | |
| 120 | 10% sucrose | — | 0.75 | ad 1 ml PBsucrose | 239 | 0.076 | |
| 100 | 10% sucrose | — | 0.625 | ad 1 ml PBsucrose | 241 | 0.073 | |
| 80 | 10% sucrose | — | 0.5 | ad 1 ml PBsucrose | 238 | 0.136 | |
| 40 | 10% sucrose | — | 0.25 | ad 1 ml PBsucrose | 235 | 0.099 | |
| 20 | 10% sucrose | — | 0.125 | ad 1 ml PBsucrose | 226 | 0.066 | |
| 160 | 10% sucrose | 12 mM phosphate | 1 | — | 278 | 0.195 | Gel |
| 160 | 10% sucrose | 24 mM phosphate | 1 | +6 μl 1.93M phosphate | 262 | 0.041 | Gel |
| 160 | 10% sucrose | 48 mM phosphate | 1 | +18 μl 1.93M phosphate | 270 | 0.017 | Gel |
| 80 | 10% sucrose | — | 0.5 | +0.5 ml PBsucrose | 236 | 0.065 | |
| 80 | 15% sucrose | — | 0.5 | +70 μl 2.4M sucr + | 206 | 0.145 | |
| | | | | 430 μl PBsucr | | | |
| 80 | 20% sucrose | — | 0.5 | +140 μl 2.4M sucr + | 186 | 0.169 | |
| | | | | 360 μl PBsucr | | | |
| 80 | 5% sucrose | — | 0.5 | +0.5 ml PBS | 244 | 0.117 | 13.4% |
| 80 | 5% sucrose + | — | 0.5 | +0.5 ml PBmaltose | 243 | 0.047 | |
| | 5% Maltose | | | | | | |
| 160 | 10% sucrose | Thaw temperature 4° C. | 1 | — | 276 | 0.103 | Gel |
| 160 | 10% sucrose | Thaw temperature 25° C. | 1 | — | 265 | 0.127 | Gel |
| 160 | 10% sucrose | Thaw temperature 37° C. | 1 | — | 262 | 0.084 | Gel |
| 160 | 10% sucrose | Extrusion 200 nm | 1 | 1 Passage | 263 | 0.183 | Gel |
| 160 | 10% sucrose | 50 mM Hepes | 1 | +50 μl 1M Hepes | 226 | 0.046 | (Gel) |
| 160 | 10% sucrose | 100 mM Hem | 1 | +100 μl 1M Hepes | 242 | 0.080 | (Gel) |
| 160 | 10% sucrose | 50 mM Tris | 1 | +50 μl 1M Tris | 205 | 0.131 | |
| 160 | 10% sucrose | 100 mM Tris | 1 | +100 μl 1M Tris | 192 | 0.126 | |
| 160 | 10% sucrose | 50 mM MOPS | 1 | +50 μl 1M MOPS | 267 | 0.061 | Gel |
| 160 | 10% sucrose | 100 mM MOPS | 1 | +100 μl 1M MOPS | 264 | 0.063 | Gel |
| 100 | 10% sucrose | — | 0.625 | ad 1 ml PBsucrose | 241 | 0.073 | |
| 120 | 10% sucrose | — | 0.75 | | 239 | 0.076 | 9.5% |
| 140 | 10% sucrose | — | 0.875 | | 249 | 0.099 | (Gel) |
| 160 | 10% sucrose | — | 1 | | 265 | 0.127 | Gel |
| 100 | 15% sucrose | — | 0.625 | +70 μl 2.4M sucrose | 200 | 0.180 | |
| 120 | 15% sucrose | — | 0.75 | ad 1 ml PBsucrose | 204 | 0.122 | |
| 140 | 15% sucrose | — | 0.875 | | 205 | 0.144 | 3.7% |
| 160 | 15% sucrose | — | 1 | | 214 | 0.137 | |
| 100 | 20% sucrose | — | 0.625 | +140 μl 2.4M sucrose | 185 | 0.120 | |
| 120 | 20% sucrose | — | 0.75 | ad 1 ml PBsucrose | 190 | 0.113 | |
| 140 | 20% sucrose | — | 0.875 | | 194 | 0.161 | 2.7% |
| 160 | 20% sucrose | — | 1 | | 199 | 0.121 | |
| 100 | 10% sucrose | 48 mM phosphate | 0.625 | | 266 | 0.087 | Gel |
| 120 | 10% sucrose | 48 mM phosphate | 0.75 | +18 μl 1.93M phosphate | 254 | 0.136 | Gel |
| 140 | 10% sucrose | 48 mM phosphate | 0.875 | ad 1 ml PBsucrose | 258 | 0.004 | Gel |
| 160 | 10% sucrose | 48 mM phosphate | 1 | | 270 | 0.017 | Gel |
| 30 | 10% trehalose | — | 0.2 | +300/450/600 μl 1M trehalose | 225 | 0.075 | |
| 30 | 15% trehalose | — | 0.2 | +100 μl 100 mM phosphate | 209 | 0.156 | |
| 30 | 20% trehalose | — | 0.2 | pH7.5 | 187 | 0.065 | 2.5% |
| | | | | ad 1 ml H2O | | | |
| 145 | 10% sucrose | 20 mM Tris | 0.9 | +20/50/75/100 μl 1M Tris | 221 | 0.049 | 5.9% |
| 145 | 10% sucrose | 50 mM Tris | 0.9 | ad 1 ml 1M Hepes | 203 | 0.050 | 7.1% |
| 145 | 10% sucrose | 75 mM Tris | 0.9 | | 192 | 0.054 | |
| 145 | 10% sucrose | 100 mM Tris | 0.9 | | 175 | 0.075 | 1.9% |
| 30 | 15% trehalose | 50 mM Tris | 0.2 | +50 μl 1M Tris + 450 μl 1M | 172 | 0.132 | 1.8% |
| | | | | trehalose + 300 μl H2O | | | |
| 80 | 10% sucrose | 100 mM Tits | 0.5 | +62/31/0 μl 2.4M sucrose + | 169 | 0.197 | 1.5% |
| 80 | 7.5% sucrose | 100 mM Tris | 0.5 | 100 μl 1M Tris pH 8 | 171 | 0.119 | 2.0% |
| 80 | 5% sucrose | 100 mM Tris | 0.5 | ad 1 ml H2O | 175 | 0.179 | 2.2% |
| 30 | 2% sucrose | — | 0.2 | 800 μl PBS | 217 | 0.150 | 7.3% |
| 160 | 10% sucrose | no freeze-thaw | 1 | Reference | 171 | 0.214 | 0.2% |

Result:

As shown in table 9, by adding sugar and/or Tris, the liposome suspension can be stabilized in such a way that size, distribution and active substance content do not substantially differ from the starting suspension.

### Example 6

### Influence of the Extrusion Step on the Liposome Quality

The liposomes as described in Example 4 were produced with the following parameters: 33 mM ethanolic lipid solution consisting of 6 mole-% POPC, 24 mole-% DOPE, 47 mole-% MoChol and 23 mole-% Chems, 30% ethanol injection, N/P=3. Dilution to 10% ethanol and rebuffering to pH 7.5 were effected in a single step with a flow rate of 160 ml/min, using a solution consisting of 136 mM sodium hydrogen phosphate and 100 mM sodium chloride, pH 9. This preparation, 50 ml each time, was further processed as follows:

1) Filtration through a 0.2 μm sterile filter

2) Extrusion through a 400 nm polycarbonate membrane

3) Extrusion through a 200 nm polycarbonate membrane and subsequent filtration through a 0.2 μm sterile filter

4) Extrusion through a 100 nm polycarbonate membrane

5) Extrusion through a 200 nm polycarbonate membrane, filtration through a 0.2 μm sterile filter and subsequent extrusion through a 100 nm polycarbonate membrane

The processed liposomes were tested for particle size, particle size distribution and lipid concentration (as in Example 4). The amount of non-entrapped active substance was separated using ultrafiltration (Centrisart, 100 kD, 2500×g, 2 hours, 20° C.) and determined by means of OD_{260 nm} against a reference solution including CD40.

Result:

TABLE 10

| | | | Poly | | |
|---|---|---|---|---|---|
| No. | Process steps | Size (nm) | index | % inside | % lipid loss |
| 1 | Shift + SF200 | 184 | 0.313 | 67% | 34% |
| 2 | Shift + Ex400 | 179 | 0.297 | 68% | 19% |
| 3 | Shift + Ex200 + | 162 | 0.213 | 65% | 6% |
| | SF200 | | | | |
| 4 | Shift + Ex100 | 154 | 0.208 | 53% | 5% |
| 5 | Shift + Ex200 + | 145 | 0.198 | 54% | 5% |
| | SF200 + Ex100 | | | | |

Extrusion can improve the quality of the liposomes in such a way that subsequent sterile filtration can be performed without significant loss of active substance and particles.

### Example 7

### Preparation of Different Nucleic Acid Loaded Amphoteric Liposomes with Defined Specifications and Comparison of a Further Processing Including Extrusion at pH 4 and pH 7.5

Different nucleic acid loaded amphoteric liposomes were produced using the following process parameters:

TABLE 11

| Parameters | Formulation 1 | Formulation 2 | Formulation 3 | Formulation 4 |
|---|---|---|---|---|
| | POPC/DOPE/ | POPC/DOPE/ | POPC/DOPE/ | POPC/DOPE/ |
| | MoChol/DMG-Succ | MoChol/Chems | MoChol/Chems | MoChol/Chems |
| | 6:24:23 :47 | 15:45:20:20 | 6:24:47:23 | 6:24:47:23 |
| N/P | 4 | 4.5 | 2 | 6 |
| Lipid conc. | 10 | 50 | 25 | 50 |
| [mM] | | | | |
| mixing ratio | 1/2.33 | 1/2.33 | 1/2.33 | 1/2.33 |
| (lipid/nucleic | | | | |
| acid | | | | |
| Loading buffer | HAc20/ | HAc20/ | HAc10/ | HAc10/ |
| pH adjusted | Sucrose 300, | Sucrose 300, | Sucrose 300, | Sucrose 300, |
| with 2M Tris | pH 4 | pH 4 | pH 4 | pH 4 |
| Shift buffer | 136 mM | 136 mM | 150 mM Tris, | 150 mM Tris, |
| | Na2HPO4/ | Na2HPO4/ | pH 7.5 | pH 7.5 |
| | 100 mM NaCl | 100 mM NaCl | | |
| Lipid solvent | Isopropanol + | Isopropanol + | Isopropanol + | Isopropanol + |
| | 10 mM HAc | 10 mM HAc | 10 mM HCl | 10 mM HCl |

Briefly, the lipid mixtures were weighed and dissolved in the appropriate lipid solvent to a desired lipid concentration. An 18mer CD40 antisense oligonucleotide was dissolved in the appropriate buffer. The amount of antisense oligonucleotide was calculated so as to obtain a defined N/P ratio in the batch (see above). The two solutions were mixed at specific flow rates in a device in accordance with FIG. 1. Following the preparation, a part of the particles were further processed as shown below.

1. Extrusion through a 400 nm polycarbonate membrane at pH 4 and 30% alcohol

2. Dilution of the mixture to 10% alcohol with the appropriate acidic buffer, Extrusion through a 400 nm polycarbonate membrane at pH 4 and 10% alcohol

3. Dilution of the mixture to 10% alcohol with the appropriate shift buffer, Extrusion through a 400 nm polycarbonate membrane at pH 7.5 and 10% alcohol

To determine the inclusion efficiency, non-entrapped antisense oligonucleotide was removed using Centriprep ultrafiltration units (100 kDa MWCO) and determined using OD measurement at 260 nm. Following dilution of the samples (max. 1% ethanol) in PBS, the size of the liposomes was determined using photon correlation spectroscopy (PCS).

Determination of the Lipid Concentration:

Following extraction into chloroform, the lipid was determined as inorganic phosphate (P. Veldhofen and G. Mannaerts (1987) Anal. Biochem. 161, 45-48).

Result:

TABLE 12

| | | | | | | Final |
|---|---|---|---|---|---|---|
| | | | | | | drug/lipid |
| | | | | | | (encapsu- |
| | | | | | % | lated drug) |
| Formu- | | Size | Poly | % | lipid | [mg drug/ |
| lation | Extrusion pH | (nm) | index | inside | loss | g lipid] |
| 1 | no extrusion | 193 | 0.049 | 44.7 | 0 | 11.3 |
| | 4 (30% alcohol) | 276 | 0.393 | 74.9 | 37.5 | ND |
| | 4 (10% alcohol) | 234 | 0.008 | 43.3 | 23.9 | ND |
| | 7.5 (10% alcohol) | 188 | 0.213 | 40.9 | 1.1 | ND |
| 2 | no extrusion | 180 | 0.281 | 91.6 | 0 | 18.7 |
| | 4 (30% alcohol) | 162 | 0.252 | 93.2 | 5.7 | ND |
| | 4 (10% alcohol) | 182 | 0.228 | 92.1 | 20.4 | ND |
| | 7.5 (10% alcohol) | 180 | 0.234 | 90.7 | 5.7 | ND |
| 3 | no extrusion | 117 | 0.491 | 71.5 | 0 | 89.1 |
| | 4 (30% alcohol) | 157 | 0.370 | 71.2 | 6.6 | ND |
| | 4 (10% alcohol) | 138 | 0.418 | 74.4 | 10.8 | ND |
| | 7.5 (10% alcohol) | 125 | 0.394 | 71.7 | 3.6 | ND |
| 4 | no extrusion | 78 | 0.216 | 76.4 | 0 | 27.8 |
| | 4 (30% alcohol) | 92 | 0.166 | 74.1 | 0 | ND |
| | 4 (10% alcohol) | 86 | 0.266 | 80.7 | 3.4 | ND |
| | 7.5 (10% alcohol) | 90 | 0.174 | 67.0 | 0 | ND |

The polydispersity indices show that an extrusion step can improve the quality of the liposomes without significant loss of active substance and lipid. An extrusion under binding conditions (pH 4) leads in most cases to a higher lipid loss than an extrusion under non-binding conditions at pH 7.5. Formulation 1 has already a very narrow size distribution before the extrusion step and subsequently a further improvement is not possible.

### Example 8

### Influence of Different Process Parameters on the Specification of Nucleic Acid Loaded Amphoteric Liposomes

a. Variation N/P Ratio

Lipid mixtures as shown below were weighed and dissolved in the appropriate lipid solvent to a desired lipid concentration. An 18mer CD40 antisense oligonucleotide was dissolved in the appropriate buffer. The amount of antisense oligonucleotide was calculated so as to obtain a defined N/P ratio in the batch (see below). The two solutions were mixed at specific flow rates in a device in accordance with FIG. 1. In a next step the mixtures were diluted to 10% alcohol with the appropriate shift buffer.

The nucleic acid loaded amphoteric liposomes were produced using the following process parameters:

TABLE 13

| Parameters | Formulation 1 | Formulation 2 | Formulation 3 |
|---|---|---|---|
| | POPC/DOPE/ | POPC/DOPE/ | POPC/DOTAP/ |
| | MoChol/Chems | MoChol/Chems | Chems |
| | 15:45:20:20 | 6:24:47:23 | 25:28:47 |
| N/P | Variation | Variation | Variation |
| | 1.75-3.5 | 2-4 | 1.3-2 |
| Lipid conc. | 20 | 20 | 25 |
| [Mm] | | | |
| mixing ratio | 1/2.33 | 1/2.33 | 1/2.33 |
| (lipid/nucleic | | | |
| acid | | | |
| Loading buffer | 20 mM HAC/ | 10 mM HAc/ | 10 mM HAc/ |
| pH adjusted | 300 mM Sucrose, | 300 mM Sucrose, | 300 mM Sucrose, |
| with 2M Tris | pH 4 | pH 4 | pH 4 |
| Shift buffer | 150 mM Tris, | 150 mM Tris, | 150 mM Tris, |
| | pH 7.5 | pH 7.5 | pH 7.5 |
| Lipid solvent | Isopropanol + | Isopropanol | Isopropanol |
| | 10 mM HAc | | |

The inclusion efficiency, the size of the liposomes and the lipid concentration was determined as described in example 7.

Results:

TABLE 14
Formulation 1:

| | | | Inclusion |
|---|---|---|---|
| N/P | Size | PI | efficiency |
| 1.75 | 114.6 | 0.320 | 85% |
| 2.5 | 115.1 | 0.276 | 83% |
| 2.75 | 101.8 | 0.275 | 85% |
| 3 | 91.2 | 0.199 | 83% |
| 3.5 | 88.1 | 0.183 | ND |

TABLE 15
Formulation 2:

| | | | Inclusion |
|---|---|---|---|
| N/P | Size | PI | effciency |
| 2 | 97.7 | 0.457 | 64% |
| 3 | 82.5 | 0.389 | 65% |
| 4 | 65.3 | 0.373 | 63% |

TABLE 16
Formulation 3:

| | | | Inclusion |
|---|---|---|---|
| N/P | Size | PI | effciency |
| 1.3 | 154.8 | 0.399 | 85% |
| 1.6 | 110.5 | 0.347 | 91% |
| 2 | 90.9 | 0.276 | 92% |

The results show that an increase of the initial N/P ratio leads to a decrease of the liposomal size. In contrast, the inclusion efficiency remains nearly unaffected by a variation of the N/P ratio.
b. lipid Concentration

Nucleic acid loaded amphoteric liposomes were produced as described under a. using the following process parameters:

TABLE 17

| Parameters | Formulation 4 |
|---|---|
| | POPC/DOPE/ |
| | MoChol/DMG-Succ |
| | 6:24:23:47 |
| N/P | 4 |
| Lipid conc. | Variation |
| [mM] | 7-35 |
| mixing ratio | 1/2.33 |
| (lipid/nucleic | |
| acid | |
| Loading buffer | 20 mM HAc/ |
| pH adjusted | 300 mM Sucrose, |
| with 2M Tris | pH 4 |
| Shift buffer | 136 mM |
| | Na2HPO4/ |
| | 100 mM NaCl |
| Lipid solvent | Isopropanol |

### Results:

TABLE 18
Formulation 4:

| Lipid | | |
|---|---|---|
| Concentration | | |
| [mM] | Size | PI |
| 7 | 237.4 | 0.157 |
| 10 | 247.3 | 0.060 |
| 20 | 394.0 | 0.097 |
| 35 | 632.9 | 0.447 |

The size of the liposomes increase with increasing lipid concentration, whereas in this concentration range no constant value was reached.

c. mixing Velocity

Nucleic acid loaded amphoteric liposomes were produced as described under a. using the following process parameters shown below. To compare different mixing velocities all formulations were prepared in the same device in accordance with FIG. 1 using different flow rates.

TABLE 19

| Parameters | Formulation 5 | Formulation 6 | Formulation 7 | Formulation 8 |
|---|---|---|---|---|
| | POPC/DOPE/ | POPC/DOPE/ | POPC/DOPE/ | POPC/DOPE/ |
| | MoChol/Chems | MoChol/Chems | MoChol/Chems | MoChol/DMG-Succ |
| | 6:24:47:23 | 6:24:47:23 | 15:45:20:20 | 6:24:23:47 |
| N/P | 4 | 5 | 1.75 | 4 |
| Lipid conc. | 20 | 50 | 20 | 20 |
| [mM] | | | | |
| mixing ratio | 1/2.33 | 1/2.33 | 1/2.33 | 1/2.33 |
| (lipid/nucleic | | | | |
| acid) | | | | |
| Flow rate | Variation | Variation | Variation | Variation |
| [ml/min] | 30/70 | 30/70 | 30/70 | 30/70 |
| (lipid/nucleic | 20/46.66 | 20/46.66 | 20/46.66 | 20/46.66 |
| acid) | 10/23.33 | 10/23.33 | 10/23.33 | 10/23.33 |
| Loading buffer | 10 mM HAc/ | 10 mM HAc/ | 10 mM HAc/ | 10 mM HAc/ |
| pH adjusted | 300 mM Sucrose, | 300 mM Sucrose, | 300 mM Sucrose, | 300 mM Sucrose, |
| with 2M Tris | pH 4 | pH 4 | pH 4 | pH 4 |
| Shift buffer | 150 mM Tris, | 150 mM Tris, | 150 mM Tris, | 150 mM Tris, |
| | pH 7.5 | pH 7.5 | pH 7.5 | pH 7.5 |
| Lipid solvent | Isopropanol | Isopropanol + | Isopropanol + | Isopropanol + |
| | | 10 mM HAc | 10 mM HAc | 10 mM HAc |

### Results:

TABLE 20
Formulation 5:

| Flow rate | | |
|---|---|---|
| [ml/min] | | |
| (lipid/nucleic acid) | Size [nm] | PI |
| 30/70 | 47.2 | 0.260 |
| 20/46.66 | 57.3 | 0.377 |
| 10/23.33 | 65.3 | 0.373 |

TABLE 21
Formulation 6:

| Flow rate | | |
|---|---|---|
| [ml/min] | | |
| (lipid/nucleic acid) | Size [nm] | PI |
| 30/70 | 91.4 | 0.270 |
| 20/46.66 | 111.3 | 0.303 |
| 10/23.33 | 171.1 | 0.420 |

TABLE 22
Formulation 7:

| Flow rate | | |
|---|---|---|
| [ml/min] | | |
| (lipid/nucleic acid) | Size [nm] | PI |
| 30/70 | 93.3 | 0.334 |
| 20/46.66 | 107.2 | 0.315 |
| 10/23.33 | 114.6 | 0.320 |

TABLE 23
Formulation 8:

| Flow rate | | |
|---|---|---|
| [ml/min] | | |
| (lipid/nucleic acid) | Size [nm] | PI |
| 30/70 | 82.2 | 0.137 |
| 20/46.66 | 93.6 | 0.095 |
| 10/23.33 | 98.8 | 0.167 |

The result shows that the size of the liposomes can be controlled by varying the mixing velocity of lipid and nucleic acid solutions and become larger by using lower mixing velocities.

d. Lipid Solvent

Nucleic acid loaded amphoteric liposomes were produced as described under a. using the following process parameters:

TABLE 24

| Parameters | Formulation 9 | Formulation 10 |
|---|---|---|
| | POPC/DOPE/ | POPC/DOPE/ |
| | MoChol/Chems | MoChol/DMG-Succ |
| | 15:45:20:20 | 6:24:23:47 |
| N/P | 1.75 | 4 |
| Lipid conc. | 20 | 10 |
| [mM] | | |
| mixing ratio | 1/2.33 | 1/2.33 |
| (lipid/nucleic | | |
| acid) | | |
| Loading buffer | 10 mM HAc/ | 20 mM HAc/ |
| pH adjusted | 300 mM Sucrose, | 300 mM Sucrose, |
| with 2M Tris | pH 4 | pH 4 |
| Shift buffer | 150 mM Tris, | 136 mM Na₂HPO₄/ |
| | pH 7.5 | 100 mM |
| | | NaCl |
| Lipid solvent | Variation | Variation |
| | Isopropanol | Isopropanol |
| | Isopropanol + | Isopropanol + |
| | 10 mM HCl | 10 mM HCl |
| | Isopropanol + | Isopropanol + |
| | 5 mM HCl | 5 mM HCl |
| | Isopropanol + | Isopropanol + |
| | 10 mM HAc | 10 mM HAc |

### Results:

TABLE 25
Formulation 9:

| Lipid solvent | Size [nm] | PI |
|---|---|---|
| Isopropanol | 72.7 | 0.167 |
| Isopropanol + 10 mM HCl | 55.0 | 0.108 |
| Isopropanol + 5 mM HCl | 60.2 | 0.159 |
| Isopropanol + 10 mM HAc | 59.0 | 0.212 |

TABLE 26
Formulation 10:

| Lipid solvent | Size [nm] | PI |
|---|---|---|
| Isopropanol | 218.9 | 0.082 |
| Isopropanol + 10 mM HCl | 104.4 | 0.209 |
| Isopropanol + 5 mM HCl | 134.3 | 0.122 |
| Isopropanol + 10 mM HAc | 175.4 | 0.057 |

This experiments shows that the size of the liposomes may be influenced also by the choice of the lipid solvent.

### Example 9

### Variation of the Ratio Cationic Lipid/Anionic Lipid

Lipid mixtures as shown below were weighed and dissolved in the appropriate lipid solvent to a desired lipid concentration. An 18mer CD40 antisense oligonucleotide was dissolved in the appropriate buffer. The amount of antisense oligonucleotide was calculated so as to obtain a defined N/P ratio in the batch (see below). The two solutions were mixed at specific flow rates in a device in accordance with FIG. 1. In a next step the mixtures were diluted to 10% alcohol with the appropriate shift buffer.

The processed liposomes were tested for particle size, particle size distribution and lipid concentration (as in Example 4). The amount of non-entrapped active substance was separated using ultrafiltration (Centrisart, 100 kD, 2500×g, 2 hours, 20° C.) and determined by means of OD_{260 nm} against a reference solution including CD40.

The nucleic acid loaded amphoteric liposomes were produced with following constant process parameters:

N/P=1.6

Lipid concentration: 25 mM
Mixing ratio (lipid/nucleic acid): 1/2.33
Loading buffer: 10 mM HAc, 300 mM Sucrose, pH 4 adjusted with 2 M Tris
Shift buffer: 150 mM Tris, pH 7.5
Lipid solvent: Isopropanol

Following formulations 1-5 were prepared using these process parameters:

### Formulation 1: POPC/DOTAP/Chems 25:22:53

### Formulation 2: POPC/DOTAP/Chems 25:25:50

### Formulation 3: POPC/DOTAP/Chems 25:28:47

### Formulation 4: POPC/DOTAP/Chems 25:31:44

### Formulation 5: POPC/DOTAP/Chems 25:34:41

### Results:

TABLE 27

| | | | | Final drug/lipid |
|---|---|---|---|---|
| | | | Encapsulation | (encapsulated drug) |
| | Size | PI | efficiency | [mg drug/g lipid] |
| Formulation 1 | 152.2 | 0.305 | 88.2 | 61.8 |
| Formulation 2 | 152.2 | 0.283 | 90.0 | 70.9 |
| Formulation 3 | 153.7 | 0.333 | 89.5 | 78.3 |
| Formulation 4 | 143.1 | 0.366 | 92.0 | 88.0 |
| Formulation 5 | 157.5 | 0.476 | 91.5 | 94.9 |

By maintaining the initial N/P ratio the final drug/lipid ratio can be increased by varying the ratio cationic to anionic lipids in the membrane of the amphoteric liposomes, whereas the size of the liposomes remains nearly constant.

### Example 10

### Lamellarity of Oligonucleotide Loaded Amphoteric Liposomes Prepared According the Method of the Present Invention

Following formulations shown below with the lipid composition POPC/DOPE/MoChol/Chems 6:24:47:23 were prepared by a 30% ethanolic injection (Loading buffer: 20 mM NaAc, 300 mM Sucrose, pH 4 adjusted with HAc; Shift buffer: 136 mM Na2HPO4, 100 mM NaCl, pH 9.2; Lipid stock solution 50 mM) or by a lipid film/extrusion process (phosphate buffered 300 mM Sucrose, pH 7.5). Formulation A was prepared as empty liposomes, all other formulations were prepared in the presence of a CD40 antisense oligonucleotide (N/P=3). Formulations C and D were extruded at pH 4. The freeze/thaw step of formulation E was performed at pH 7.5. Formulation F was prepared by a passive loading process at pH 7.5 using a lipid film/extrusion process. After the preparation all formulations were dialyzed against phosphate buffered 300 mM Sucrose, pH 7.5.

TABLE 28

| | | Injection | | | |
|---|---|---|---|---|---|
| Sample | Manufacturing | pH | Extrusion | Size [nm] | PI |
| A | Injection | 4.0 | — | 112.6 | 0.248 |
| | (empty) | | | | |
| B | Injection | 4.0 | — | 178.0 | 0.172 |
| C | Inj + Extrusion | 4.0 | pH 4; | 126.0 | 0.114 |
| | (30%) | | 30% EtOH; | | |
| | | | 15x 200/200 | | |
| D | Inj + Extrusion | 4.0 | pH 4; | 141.7 | 0.115 |
| | (10%) | | 10% EtOH; | | |
| | | | 15x 200/200 | | |
| E | Injection + | 4.0 | — | 172.2 | 0.153 |
| | Freeze-thaw | | | | |
| | (pH 7.5) | | | | |
| F | Lipid film | — | pH 7.5; 15x | 180.3 | 0.097 |
| | | | 200/200 | | |

The lamellarity of all liposomal formulations was determined by Small Angle X-Ray Scattering (SAXS).

The results are shown below:

TABLE 29

| | | | Lipid concentration |
|---|---|---|---|
| Sample | Manufacturing | Lamellarity | [mM] |
| A | Injection (empty) | oligo | 118 |
| B | Injection | multi | 75 |
| C | Inj + Extrusion | multi | 71 |
| | (30%) | | |
| D | Inj + Extrusion | multi | 102 |
| | (10%) | | |
| E | Injection + | multi | 81 |
| | Freeze-thaw | | |
| F | Lipid film | uni/oligo | 86 |

The results in table 29 show that all liposomal formulations encapsulating CD40 antisense oligonucleotides prepared by the method according to the present invention (formulations B-E) are multilamellar. In contrast, liposomes prepared by the passive loading process are unilamellar and/or oligolamellar (formulation F). Empty liposomes prepared by the ethanol injection method are oligolamallar (formulation A).

### Example 11

### Plasmid Loaded Amphoteric Liposomes Prepared According the Method of the Present Invention

Liposomes were produced by injecting 10 Vol-% of an ethanolic lipid solution (20 mM Lipid) into 10 mM NaAc, pH 4.5 adjusted with HAc, containing a 7000 bp plasmid encoding for luciferase. The resulting lipid concentration was 2 mM. The pH of this solution was immediately shifted with 1/10 volume 1M Hepes pH 8. To concentrate the diluted liposomes the suspensions were sedimented for 1 h at 80.000 rpm in a TLA 100.4 rotor (Beckman Optima-MAX). The amount of plasmid in the aqueous phase was calculated to obtain the desired N/P ratios as shown below.

One formulation (Formulation C3) was prepared by injecting 30 Vol-% of an ethanolic lipid solution (20 mM) into 10 mM NaAc, pH 4.5 adjusted with HAc, containing a 7000 bp plasmid encoding for luciferase. The mixture was then diluted with the acidic buffer to a resulting lipid concentration of 2 mM. The pH of this solution was immediately shifted with 1/10 volume 1M Hepes pH 8.

Following dilution of the samples (max. 1% ethanol) in PBS, the size of the liposomes was determined using photon correlation spectroscopy (PCS).

To determine the encapsulation efficiency non-encapsulated plasmid was removed. Therefore the concentrated liposomal suspensions were diluted with a sucrose stock solution and brought to 0.8M sucrose. 0.5M sucrose in PBS and pure PBS were layered on top, forming a gradient for removing the plasmid outside of the particles. Sucrose gradients were spun for 45 min at 40.000 rpm in a MLS-50 rotor (Beckman Optima-MAX) and the liposomes were taken from the upper interphase.

Determination of the Lipid Concentration:

Following extraction into chloroform, the lipid was determined as inorganic phosphate (P. Veldhofen and G. Mannaerts (1987) Anal. Biochem. 161, 45-48).

Determination of Plasmid Concentration:

The amount of encapsulated plasmid was determined using PicoGreen dsDNA Quantitation Reagent (Invitrogen).

TABLE 30

| No. | Formulation | N/P |
|---|---|---|
| A1 | POPC/DOPE/MoChol/DOG-Succ | 36.7 |
| | 10:40:33:17 | |
| A2 | POPC/DOPE/MoChol/DOG-Succ | 18.8 |
| | 10:40:33:17 | |
| B1 | POPC/DOPE/MoChol/DOG-Succ | 25.6 |
| | 7.5:22.5:23:47 | |
| B2 | POPC/DOPE/MoChol/DOG-Succ | 12.8 |
| | 7.5:22.5:23:47. | |
| C1 | DOPE/MoChol/DMG-Succ | 14.75 |
| | 50:30:20 | |
| C2 | DOPE/MoChol/DMG-Succ | 7.4 |
| | 50:30:20 | |
| C3 | DOPE/MoChol/DMG-Succ | 7.4 |
| | 50:30:20 | |

Results:

The size and the encapsulation efficiency of the different plasmid loaded amphoteric liposomes are shown in table 31 below.

TABLE 31

| | | | Encapsulation |
|---|---|---|---|
| No. | Size [nm] | PI | efficiency [%] |
| A1 | 130 | 0.212 | 27 |
| A2 | 172 | 0.356 | 25 |
| B1 | 193 | 0.231 | 64 |
| B2 | 206 | 0.312 | 68 |
| C1 | 128 | 0.304 | 41 |
| C2 | 221 | 0.374 | 60 |
| C3 | 225 | 0.315 | 76 |

Table 31 shows that the size of the plasmid loaded liposomes increase with lowering the N/P ratio, whereas the encapsulation efficiency remains nearly constant. A comparison of formulation C2 and C3 shows that the encapsulation efficiency can be increased by using higher amounts of ethanol in the mixture and following dilution.

### Example 12

### Lamellarity of Plasmid Loaded Amphoteric Liposomes Prepared According the Method of the Present Invention

Liposomes with the lipid composition DOPE/MoChol/Chems 50:20:30 were produced by injecting 10 Vol-% of an ethanolic lipid solution (20 mM Lipid) into 10 mM NaAc, pH 4.5 adjusted with HAc, containing a plasmid encoding for GFP. The resulting lipid concentration was 2 mM. The pH of this solution was immediately shifted with 1/10 volume 1M Hepes pH 8. To concentrate the diluted liposomes the suspensions were sedimented for 1 h at 80.000 rpm in a TLA 100.4 rotor (Beckman Optima-MAX).

Following dilution of the samples (max. 1% ethanol) in PBS, the size of the liposomes was determined using photon correlation spectroscopy (PCS).

The lamellarity of the plasmid loaded liposomal formulation was determined by Small Angle X-Ray Scattering (SAXS).

### Results:

| | |
|---|---|
| Formulation: | DOPE/MoChol/Chems 50:20:30 |
| Size: | 197 nm |
| PI: | 0.269 |
| Lipid concentration: | 120 mM |

The SAXS measurements showed that the plasmid loaded amphoteric liposomes are oligolamellar.

1. A method for preparing amphoteric liposomes loaded with a polyanionic active agent as cargo, characterised by providing an aqueous solution of said polyanionic active agent and an alcoholic solution of one or more amphiphiles wherein at least one of said solutions requiring adjustment to an acidic pH and admixing said solutions, said one or more amphiphiles being susceptible of forming amphoteric liposomes at said acidic pH, thereby to form such amphoteric liposomes in suspension encapsulating said active agent under conditions such that said liposomes form aggregates, and thereafter treating said suspension to dissociate said aggregates.

2. The method as claimed in claim 1, wherein said acidic pH is at least one unit lower than the isoelectric point of said one or more of amphiphiles.

3. The method as claimed in claim 1, wherein said alcoholic solution is buffered to an acidic pH using a buffer selected from acetate buffers, formiate buffers, glycine buffers, maleic acid buffers, phosphate buffers and citrate buffers or an acid selected from HCl, acetic acid, formic acid, maleic acid, sulfonic acid, phosphoric acid and citric acid.

4. The method as claimed in claim 1, wherein said aqueous solution is buffered to an acidic pH using a buffer selected from acetate buffers, formiate buffers, glycine buffers, maleic acid buffers, phosphate buffers and citrate buffers or an acid selected from HCl, acetic acid, formic acid, maleic acid, sulfonic acid, phosphoric acid and citric acid.

5. The method as claimed in claim 1, wherein said treatment to dissociate said aggregates comprises elevating the pH of the suspension to pH 7 or more or comprises elevating the ionic strength of said suspension.

6. The method as claimed in claim 1, wherein the alcohol content of said suspension after said admixing step is reduced by dilution with additional aqueous media.

7. The method as claimed in claim 6, wherein said dilution step is performed with an aqueous buffer solution adapted to elevate the pH of said suspension to pH 7 or more.

8. The method as claimed in claim 5, wherein the pH of said suspension is elevated using a buffer or base essentially comprising Irishydroxymethylaminomethan, BIS-TRIS, Carbonate, Triethanolamine, Triethylamine, Arginine, L-Arginine, Imidazole, hydrogenphosphate, HEPES buffers or NaOH.

9. The method as claimed in claim 6, wherein the alcohol content is reduced by admixing said aqueous and alcoholic solutions such that the resultant alcohol content is greater than about 25% vol. and thereafter diluting said suspension with additional aqueous media such that the alcohol content thereof is less than about 25% vol, preferably less than about 15% vol., preferably 10% vol. or less.

10. The method as claimed in claim 1, wherein said alcoholic solution comprises one or more counterions of said amphiphiles therein, which counterions are selected from carbonate, hydrogencarbonate, formiate, acetate, propionate, butyrate, isobutyrate, trimethylammonium, triethylammonium, triethanolammonium, trishydroxymethylaminomethanium, BIS-TRIS cations, imidazolium, argininium, L-argininium, phosphate, sulphate, methanesulfonate, chloride, sodium and potassium.

11. The method as claimed in claim 5, wherein the ionic strength of said suspension is increased by the addition of one or more salts thereto, which salts are selected from sodium chloride, sodium citrate and sodium phosphate.

12. The method as claimed in claim 1, further comprising extruding said liposomes in suspension at said neutral pH or by extruding said liposomes at said acidic pH.

13. The method as claimed in claim 1, further comprising freezing and thawing the liposomes in suspension at said neutral pH and optionally repeating said freezing and thawing step to obtain liposomes having a desired size distribution.

14. The method as claimed in claim 13, wherein said suspension comprises a cryoprotectant and/or a large cationic counter-ion selected from tris(hydroxymethyl)aminomethane, arginine, triethanolamine, morpholine and piperazine or sodium during said freezing and thawing step(s).

15. The method as claimed in claim 1, further comprising controlling the amount of active agent encapsulated by said liposomes by adjusting the concentration of amphiphiles in said mixture of alcoholic and aqueous solutions, adjusting the amount of alcoholic lipid solution mixed into the aqueous nucleic acid solution, adjusting the temperature at which the alcoholic and aqueous solutions are admixed, adding non-ionic ingredients to the admixture or adding ionic species to the admixture.

16. The method as claimed in claim 1, further comprising controlling the size of the amphoteric liposomes by adjusting the concentration of amphiphiles in said mixture of alcoholic and aqueous solutions, adjusting the turbulence of the admixture, or adjusting the ratio of cationic charges in said amphiphiles to anionic charges in said polyanionic active agent or adding ionic species to the admixture

17. The method as claimed in claim 16, wherein said ratio of cations in said amphiphiles to anions in the negatively charged active agent is in the range 1-10 or 1-50.

18. The method as claimed in claim 1, wherein said alcoholic solution comprises one or more alcohols selected from ethanol, isopropanol, 1,2-propane-diol, npropanol, as well as ethylene glycol, propylene glycol and methanol.

19. The method as claimed in claim 1, characterised in that said polyanionic active agent comprises a nucleic acid.

20. The method as claimed in claim 19, wherein said nucleic acid comprises nucleic acids encoding one or more specific sequences for proteins, polypeptides or RNAs or oligonucleotides that can specifically regulate protein expression levels or affect the protein structure through inter alia interference with splicing and artificial truncation.

21. A nucleic acid loaded amphoteric liposome produced by the method as claimed in claim 1, wherein said nucleic acids comprise oligonucleotides DNA plasmids, linear DNA constructs, RNA, aptamers or ribozymes with a chain length of more than 50 nucleobases.

22. The nucleic acid loaded amphoteric liposome as claimed in claim 21, wherein the size of said liposomes is between of 70 and 150 nm and the final nucleic acid/lipid ratio of said liposome is between 1 and 40 mg nucleic acid per g lipid or between 40 and 120 mg nucleic acid per g lipid.

23. The nucleic acid loaded amphoteric liposome as claimed in claim 21, wherein the size of said liposomes is between 130 and 200 nm and the final nucleic acid/lipid ratio of said liposomes is between 1 and 40 mg nucleic acid per g lipid or between 40 and 120 mg nucleic acid per g lipid.

24. The nucleic acid loaded amphoteric liposome produced by the method as claimed in claim 1, wherein the size of said liposomes is between 70 and 300 nm and the final nucleic acid/lipid ratio of said liposomes is between 0.3 and 30 mg of nucleic acid per g of lipid.

25. The nucleic acid loaded amphoteric liposome as claimed in claim 21, wherein said liposomes comprises a cryoprotectant selected from sucrose, trehalose and maltose and/or cations selected from the group comprising tris(hydroxymethyl)aminomethane, triethanolamine, morpholine, piperazine, arginine or sodium.

26. The nucleic acid loaded amphoteric liposome as claimed in claim 21, wherein said amphoteric liposomes has an isoelectric point between about 4 and about 7.4.

27. The nucleic acid loaded amphoteric liposome as claimed in claim 21, wherein said amphoteric liposomes may be formed from a lipid phase comprising one or more amphoteric lipids or wherein said amphoteric liposomes may be formed from a lipid phase comprising one or more or a plurality of charged amphiphiles which in combination with one another have amphoteric character.

28. The nucleic acid loaded amphoteric liposome as claimed in claim 27 wherein said charged amphiphiles comprise (i) a chargeable anionic lipid and a chargeable cationic lipid, (ii) a stable cationic lipid and a chargeable anionic lipid, (iii) a stable anionic lipid and a chargeable cationic lipid, or (iv) a pH sensitive anionic lipid and a pH sensitive cationic lipid.

29. The nucleic acid loaded amphoteric liposome as claimed in claim 27 wherein said cations are selected from the group comprising DPIM, DOIM, CHIM, DORIE, DDAB, DAC-Chol, TC-Chol, DOTMA, DOGS, (C18)₂Gly⁺-N,N-dioctadecylamidoglycine, CTAB, CPyC, DODAP and DOEPC, DMTAP, DPTAP, DOTAP, DC-Chol, MoChol and HisChol and/or wherein said anionic lipids are selected from the group comprising DOGSucc, POGSucc, DMGSucc, DPGSucc, DMPS, DPPS, DOPS, POPS, DMPG, DPPG, DOPG, POPG, DMPA, DPPA, DOPA, POPA, CHEMS and Cetyl-P.

30. The nucleic acid loaded amphoteric liposome as claimed in claim 21 wherein said amphoteric liposomes may be formed from a lipid phase further comprising one or more neutral lipids.

31. The nucleic acid loaded amphoteric liposome as claimed in claim 30 wherein said one or more neutral lipids are selected from the group comprising DMPC, DPPC, DSPC, POPC, DOPC, DMPE, DPPE, DSPE, POPE, DOPE, Diphythanoyl-PE, sphingomyelein, ceramide and cholesterol.

32. The nucleic acid loaded amphoteric liposome as claimed in claim 21, wherein said oligonucleotide is a decoy oligonucleotide, an antisense oligonucleotide, a siRNA, an agent influencing transcription, an agent influencing splicing, Ribozymes, DNAzymes or Aptamers.

33. The nucleic acid loaded amphoteric liposome as claimed in claim 32 wherein said oligonucleotides comprises naturally occurring or modified nucleosides such as DNA, RNA, locked nucleic acids (LNA's), 2′O-methyl RNA (2′Ome), 2′Fluoro RNA (2′F), 2′ O-methoxyethyl RNA (2′MOE) in their phosphate or phosphothioate forms or Morpholinos or peptide nucleic acids (PNA's).

34. The nucleic acid loaded amphoteric liposome as claimed in claim 32 wherein said oligonucleotide is an antisense oligonucleotide of 8 to 50 nucleotides length, a single stranded or double stranded siRNA of 15 to 30 nucleotides length, a decoy oligonucleotide of 15 to 30 nucleotides length, an agent influencing the transcription of 15 to 30 nucleotides length, a DNAzyme of 25 to 50 nucleotides length, a Ribozyme of 25 to 50 nucleotides length or an Aptamer of 15 to 60 nucleotides length.

35.-39. (canceled)

40. The nucleic acid loaded amphoteric liposomes as claimed in claim 27 wherein said amphoteric lipids are selected from Hist-Chol, HistDG, isoHistSuccDG, Acylcarnosin, HCChol, Hist-PS and EDTA-Choi.

41.-58. (canceled)
